(19) United States
(12) Patent Application Publication
(10) Pub. No.: US 2024/0062917 A1
(43) Pub. Date: Feb. 22, 2024

(54) SYSTEMS AND METHODS FOR ADMINISTERING SAFETY INFORMATION FOR HAZARDOUS DRUGS

(71) Applicant: Rpharmy, LLC, Austin, TX (US)

(72) Inventors: John Paxton, Austin, TX (US); Laura Paxton, Austin, TX (US); Daniel Curtis Robertson, Leander, TX (US); Patrick Turner, Winston-Salem, NC (US)

(21) Appl. No.: 17/821,408

(22) Filed: Aug. 22, 2022

(51) Int. Cl. G16H 70/40

(52) U.S. Cl. CPC G16H 70/40

(57) ABSTRACT

A hazardous drug administrative server includes a memory and a processor operably coupled to the memory. The memory is configured to store a hazardous drug data structure. The processor is configured to monitor hazardous drug databases for updates to the hazardous drug data structure. The processor is also configured to update the hazardous drug data structure based on the updates from the monitored hazardous drug databases. The processor is further configured to identify hospital hazardous drug data structures that are linked to the hazardous drug data structure. The processor is additionally configured to transmit the updates to the hazardous drug data structure that correspond to the identified hospital hazardous drug data structures to a hospital server.

### TECHNICAL FIELD

This disclosure relates generally to hazardous drug administrative devices and processes. More specifically, this disclosure relates to systems and methods for administering safety information for hazardous drugs at hospitals or other medical care facilities.

### BACKGROUND

Hospitals consume hazardous materials and produce hazardous waste in the ordinary course of business. Federal, state, and local regulations provide specific requirements for the handling, storage, and disposal of hazardous materials and hazardous waste. Many hospitals have higher or different standards implemented for hazardous materials and hazardous waste. As many doctors and staff can be admitted to multiple hospitals, specific requirements for hazardous materials and hazardous waste can be difficult to remember or look up to determine the proper procedures for hazardous material and hazardous waste, which may lead to injury, fines, or damage to property when handled improperly.

### SUMMARY

This disclosure provides systems and methods for administering safety information for hazardous drugs.

In a first embodiment, an apparatus includes hazardous drug administrative server includes a memory and a processor operably coupled to the memory. The memory is configured to store a hazardous drug data structure. The processor is configured to monitor hazardous drug databases for updates to the hazardous drug data structure. The processor is also configured to update the hazardous drug data structure based on the updates from the monitored hazardous drug databases. The processor is further configured to identify hospital hazardous drug data structures that are linked to the hazardous drug data structure. The processor is additionally configured to transmit the updates to the hazardous drug data structure that correspond to the identified hospital hazardous drug data structures to a hospital server.

In a second embodiment, a method includes storing a hazardous drug data structure. The method also includes monitoring hazardous drug databases for updates to the hazardous drug data structure. The method further includes updating the hazardous drug data structure based on the updates from the monitored hazardous drug databases. In addition, the method includes identifying hospital hazardous drug data structures that are linked to the hazardous drug data structure. The method also includes transmitting the updates to the hazardous drug data structure that correspond to the identified hospital hazardous drug data structures to a hospital server.

In a third embodiment, a hazardous drug system includes hazardous drug databases and a hazardous drug administrative server communicatively coupled to the hazardous drug databases. The hazardous drug databases are configured to store hazardous drug information. The hazardous drug administrative server is configured to store a hazardous drug data structure. The hazardous drug administrative server is also configured to monitor hazardous drug databases for updates to the hazardous drug data structure. The hazardous drug administrative server is configured to update the hazardous drug data structure based on the updates from the monitored hazardous drug databases. The hazardous drug administrative server is configured to identify hospital hazardous drug data structures that are linked to the hazardous drug data structure. The hazardous drug administrative server is configured to transmit the updates to the hazardous drug data structure that correspond to the identified hospital hazardous drug data structures to a hospital server.

Other technical features may be readily apparent to one skilled in the art from the following figures, descriptions, and claims.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates an example system for administering safety information for hazardous drugs in accordance with this disclosure;

FIG. 2 illustrates an example system of hardware implemented as a computing device for administering hazardous drug records in accordance with this disclosure;

FIGS. 3A through 3L illustrate an example hazardous drug data structure in accordance with this disclosure;

FIGS. 4A through 4C illustrate an example hazardous drug file setup in accordance with this disclosure;

FIGS. 5A through 5H illustrate an example hazardous drug user interface in accordance with this disclosure; and

FIG. 6 illustrates an example method for administering hazardous drug records according to this disclosure.

### DETAILED DESCRIPTION

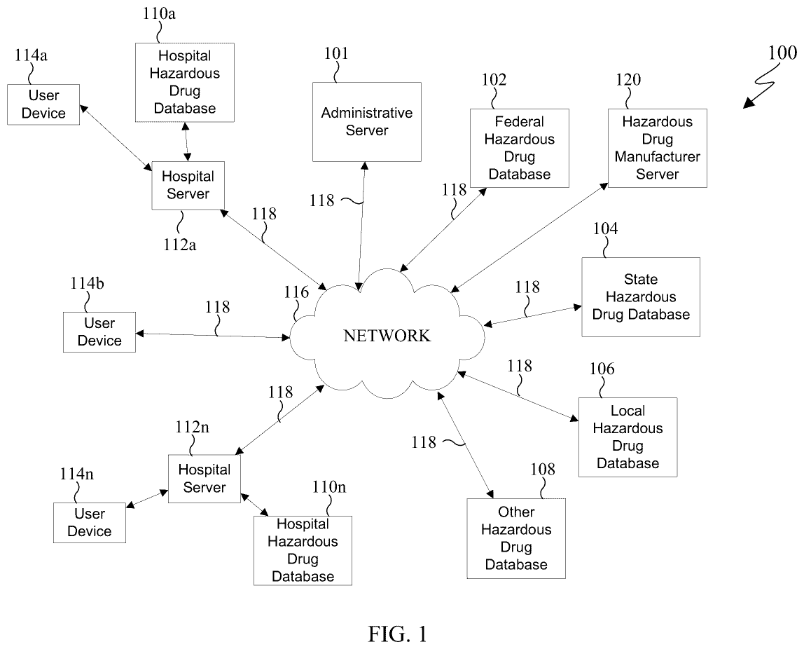

FIGS. 1 through 6, described below, and the various embodiments used to describe the principles of the present disclosure are by way of illustration only and should not be construed in any way to limit the scope of the disclosure. Those skilled in the art will understand that the principles of the present disclosure may be implemented in any type of suitably arranged device or system.

The United States Pharmacopeia (USP) is a pharmacopeia, that is, a compendium of drug information for the United States published annually by the United States Pharmacopeia Convention also called the “USP.” If a drug ingredient or drug product has an applicable USP quality standard (in the form of a USP-national formulary (NF) monograph), it must conform in order to use the designation “USP” or “NF.” Drugs subject to USP standards include both human drugs (prescription, over-the-counter, or otherwise) and also animal drugs.

USP establishes written and physical standards for medicines, food ingredients, dietary supplement products, and other products and ingredients. These standards are used by regulatory agencies and manufacturers to help to ensure that these products are of the appropriate identity, as well as strength, quality, purity, and consistency.

USP General Chapter 800 (“Chapter 800”) describes requirements for the responsibilities of health care professionals handling drugs—especially hazardous drugs. Chapter 800 also describes facility and engineering controls; procedures for deactivating, decontaminating and cleaning; spill control; and documentation of drugs. These standards apply to all healthcare personnel who receive, prepare, administer, transport or otherwise interact with hazardous drugs and all the environments in which they are handled.

Despite the requirements under Chapter 800, many health care professionals are still unaware of the risks of the drugs they are handling, administering, and supervising. As such, there is an ongoing need for methods, systems, products and other forms of administration of regulated safety requirements for health care professionals according to embodiments of the present invention described in detail below.

Some states and localities have additional requirements or instructions for handling, storing, or disposal of hazardous drugs. For example, cities may have specific or additional requirements for disposing of hazardous drugs in a city dump.

The administrative system provides an improvement to conventional, prior art computer systems by combining different information from multiple sources to reduce network complexity and processing power required for administering hazardous drugs.

The administrative system also provides an improvement to conventional, prior art computer systems by storing localized profiles specific for a hospital or medical care facility on a local server. The files are linked to the client server and can be updated periodically in case of network crash or lost internet connection.

FIG. 1 illustrates an example administrative system 100 for administering safety information for hazardous drugs in accordance with this disclosure. The embodiment of the administrative system 100 illustrated in FIG. 1 is for illustration only. FIG. 1 does not limit the scope of this disclosure to any particular implementation of an administrative system.

As shown in FIG. 1, the administrative system 100 is illustrated as one example of system hardware for managing safety information for hazardous drugs that can be used in accordance with some embodiments of the disclosed subject matter. As illustrated, the administrative system 100 can include one or more: administrative servers 101, federal hazardous drug database servers 102, state hazardous drug database servers 104, local hazardous drug database servers 106, other hazardous drug database servers 108, hospital servers 110a-110n with hospital hazardous drug databases 112a-112n, and user devices 114a-114n. In the description of FIG. 1, the administrative servers 101, federal hazardous drug database servers 102, state hazardous drug database servers 104, local hazardous drug database servers 106, other hazardous drug database servers 108 can be respectively referred to as administrative databases 101, federal hazardous drug database databases 102, state hazardous drug database databases 104, local hazardous drug database databases 106, other hazardous drug database databases 108.

In certain embodiments, the administrative server 101 and the hospital servers 112a-112n can be any suitable server for storing data and/or delivering the data to a user device 114a-114n. In certain embodiments, the data stored by the administrative server 101 and the hospital servers 112a-112n and/or delivered to the user devices 114a-114n can be implemented as digital data in any digital data format. For example, the administrative server 101 and the hospital servers 112a-112n can be implemented as a server that delivers data to a user devices 114a-114n and/or receives data from a hazardous drug database 102-110n via a communication network 116. In some embodiments, the administrative server 101 and the hospital servers 112a-112n can include a server computing device (e.g., electronic device 200) having a server communication interface operatively connected to the communication network 116 to receive respective hazardous drug data from one or more hazardous drug databases 102-110n and operatively connected to the server computing device to provide the received hazardous drug storage and handling data to the server computing device and a server memory disposed at the respective data server location and operatively connected to the server computing device for storing the received hazardous drug storage and handling data. In some embodiments, the administrative server 101 and the hospital servers 112a-112n can include a server computing device 200 having a server communication interface operatively connected to the communication network 116 to receive hazardous drug storage and handling data from one or more hazardous drug databases 102-110n and operatively connected to the server computing device to provide the received hazardous drug storage and handling data to the server computing device and a server memory disposed at the respective data server location and operatively connected to the server computing device for storing the received hazardous drug storage and handling data. Data stored and/or delivered by the administrative server 101 and the hospital servers 112a-112n can be any suitable data, such as hazardous drug data, user data, client drug data, client drug selectable data, changelog data, client data, selectable data, safety data sheet (SDS) data, IP address data, file data, multum drug data, and/or any other suitable data. Data can be updated and downloaded to the administrative server 101 and the hospital servers 112a-112n from any suitable entity (e.g., administrative server 101, federal hazardous drug databases 102, state hazardous drug databases 104, local hazardous drug databases 106, other hazardous drug databases 108, hospital hazardous drug databases 110, user devices 114 with certain level of authorization, etc.). In some other embodiments, the hospital servers 112a-112n can be omitted.

In various embodiments, the communication network 116 can be any suitable combination of one or more wired and/or wireless networks. For example, the communication network 116 can include anyone or more of the Internet, an intranet, a wide-area network (WAN), a local-area network (LAN), a wireless network, a digital subscriber line (DSL) network, a frame relay network, an asynchronous transfer mode (ATM) network, a virtual private network (VPN), and/or any other suitable communication network. The user devices 114a-114n can be connected by one or more communications links 118 to the communication network 116, which can be linked via one or more communications links 118 to the administrative server 101 and the hospital servers 112a-112n. The communications links 118 can be any communications links suitable for communicating data among the administrative server 101, the federal hazardous drug databases 102, the state hazardous drug databases 104, the local hazardous drug databases 106, the other hazardous drug databases 108, and the hospital servers 112a-112n, such as network links, dial-up links, wireless links, hard-wired links, any other suitable communications links, or any suitable combination of such links. In some embodiments, the data communicated across the communication network 116 and/or communication links 118 can be implemented as digital data in any digital data format.

The user devices 114a-114n can include any one or more user devices suitable for requesting data, searching for data, viewing data, retransmitting data, manipulating data, receiving a user input and/or any other suitable functions. For example, in some embodiments, the user devices 114a-114n can be implemented as a mobile device, such as a mobile phone, a tablet computer, a laptop computer and/or any other suitable mobile device. As another example, the user devices 114a-114n can be implemented as a non-mobile device such as a desktop computer and/or any other suitable non-mobile device. In some embodiments, the user devices 114a-114n can be disposed at a geographic location that is remote from (i.e., geographically distant from) the hospital servers 112a-112n and receive data over the network 116, whereas in other embodiments, the user devices 114a-114n can be disposed at the same geographic location as the hospital servers 112a-112n and receive data over the hospital servers 112a-112n.

In some embodiments, the federal hazardous drug database 102 can be any suitable database for storing data regarding hazardous drugs at a federal level or related to federal rules for hazardous drugs. For example, the federal hazardous drug database 102 can be a database that stores any suitable data, such as hazardous drug data, user data, client drug data, client drug selectable data, changelog data, client data, selectable data, safety data sheet (SDS) data, IP address data, file data, multum drug data, and/or any other suitable data. The administrative server 101 and the hospital servers 112a-112n can communicate with the federal hazardous drug database 102 via a communication network 116. The storage of the federal hazardous drug data, user data, client drug data, client drug selectable data, changelog data, client data, selectable data, safety data sheet (SDS) data, IP address data, file data, multum drug data and other information, programs, data and/or other suitable information on the administrative server 101, the federal hazardous drug database, and the hospital hazardous drug databases 110a-110n can be implemented as digital data in any digital data format. In some embodiments, the federal hazardous drug database 102 can include a federal hazardous drug server computing device (e.g., hardware 200) having a federal hazardous drug server communication interface operatively connected to the communication network 116 to receive hazardous drug updates from a hazardous drug manufacturer server 120 and operatively connected to the administrative server 101 to provide the received hazardous drug updates to the hospital hazardous drug database 110a-110n, and/or a hospital server memory operatively connected to the hospital server 112a-112n for storing the received hazardous drug updates. In some embodiments, the administrative computing device of the administrative server 101 can determine if a received hazardous drug update is applicable to a hazardous drug information stored on each of the hospital hazardous drug databases 110a-110n, and if so, the administrative server 101 can trigger a hazardous drug update at respective hospital hazard drug databases 110a-110n or the hospital server memory of the hospital server 112a-112n by communicating over the communication network 116.

In some embodiments, the federal hazardous drug database 102 can include a federal hazardous drug server computing device (e.g., hardware 200) having a federal hazardous drug server input/output interface to receive hazardous drug changes from a federal official with authorization to change the federal hazardous drug data and a federal hazardous drug server communication interface operatively connected to the communication network 116 operatively connected to the administrative server 101 to provide the received hazardous drug changes to the hospital hazardous drug database 110a-110n, and/or a hospital server memory operatively connected to the hospital server 112a-112n for storing the received hazardous drug changes. In some embodiments, the administrative computing device of the administrative server 101 can determine if a received hazardous drug change is applicable to a hazardous drug information stored on each of the hospital hazardous drug databases 110a-110n, and if so, the administrative server 101 can trigger a hazardous drug changes at respective hospital hazard drug databases 110a-110n or the hospital server memory of the hospital server 112a-112n by communicating over the communication network 116.

In some embodiments, the state hazardous drug database 104 can be any suitable database for storing data regarding hazardous drugs at a state level or related to state rules for hazardous drugs. For example, the state hazardous drug database 104 can be a database that stores any suitable data, such as hazardous drug data, user data, client drug data, client drug selectable data, changelog data, client data, selectable data, safety data sheet (SDS) data, IP address data, file data, multum drug data, and/or any other suitable data. The administrative server 101 and the hospital servers 112a-112n can communicate with the state hazardous drug database 104 via a communication network 116. The storage of the state hazardous drug data, user data, client drug data, client drug selectable data, changelog data, client data, selectable data, safety data sheet (SDS) data, IP address data, file data, multum drug data and other information, programs, data and/or other suitable information on the administrative server 101, the state hazardous drug database, and the hospital hazardous drug databases 110a-110n can be implemented as digital data in any digital data format. In some embodiments, the state hazardous drug database 104 can include a state hazardous drug server computing device (e.g., hardware 200) having a state hazardous drug server communication interface operatively connected to the communication network 116 to receive hazardous drug updates from a hazardous drug manufacturer server 120 and operatively connected to the administrative server 101 to provide the received hazardous drug updates to the hospital hazardous drug database 110a-110n, and/or a hospital server memory operatively connected to the hospital server 112a-112n for storing the received hazardous drug updates. In some embodiments, the administrative computing device of the administrative server 101 can determine if a received hazardous drug update is applicable to a hazardous drug information stored on each of the hospital hazardous drug databases 110a-110n, and if so, the administrative server 101 can trigger a hazardous drug update at respective hospital hazard drug databases 110a-110n or the hospital server memory of the hospital server 112a-112n by communicating over the communication network 116.

In some embodiments, the state hazardous drug database 104 can include a state hazardous drug server computing device (e.g., hardware 200) having a state hazardous drug server input/output interface to receive hazardous drug changes from a state official with authorization to change the state hazardous drug data and a state hazardous drug server communication interface operatively connected to the communication network 116 operatively connected to the administrative server 101 to provide the received hazardous drug changes to the hospital hazardous drug database 110a-110n, and/or a hospital server memory operatively connected to the hospital server 112a-112n for storing the received hazardous drug changes. In some embodiments, the administrative computing device of the administrative server 101 can determine if a received hazardous drug change is applicable to a hazardous drug information stored on each of the hospital hazardous drug databases 110a-110n, and if so, the administrative server 101 can trigger hazardous drug changes within respective hospital hazard drug databases 110a-110n or the hospital server memory of the hospital server 112a-112n by communicating over the communication network 116.

In some embodiments, the local hazardous drug database 106 can be any suitable database for storing data regarding hazardous drugs at a local level or related to local rules for hazardous drugs. For example, the local hazardous drug database 106 can be a database that stores any suitable data, such as hazardous drug data, user data, client drug data, client drug selectable data, changelog data, client data, selectable data, safety data sheet (SDS) data, IP address data, file data, multum drug data, and/or any other suitable data. The administrative server 101 and the hospital servers 112a-112n can communicate with the local hazardous drug database 106 via a communication network 116. The storage of the local hazardous drug data, user data, client drug data, client drug selectable data, changelog data, client data, selectable data, safety data sheet (SDS) data, IP address data, file data, multum drug data and other information, programs, data and/or other suitable information on the administrative server 101, the local hazardous drug database, and the hospital hazardous drug databases 110a-110n can be implemented as digital data in any digital data format. In some embodiments, the local hazardous drug database 106 can include a local hazardous drug server computing device (e.g., hardware 200) having a local hazardous drug server communication interface operatively connected to the communication network 116 to receive hazardous drug updates from a hazardous drug manufacturer server 120 and operatively connected to the administrative server 101 to provide the received hazardous drug updates to the hospital hazardous drug database 110a-110n, and/or a hospital server memory operatively connected to the hospital server 112a-112n for storing the received hazardous drug updates. In some embodiments, the administrative computing device of the administrative server 101 can determine if a received hazardous drug update is applicable to hazardous drug information stored on each of the hospital hazardous drug databases 110a-110n, and if so, the administrative server 101 can trigger a hazardous drug update at respective hospital hazard drug databases 110a-110n or the hospital server memory of the hospital server 112a-112n by communicating over the communication network 116.

In some embodiments, the local hazardous drug database 106 can include a local hazardous drug server computing device (e.g., hardware 200) having a local hazardous drug server input/output interface to receive hazardous drug changes from a local official with authorization to change the local hazardous drug data and a local hazardous drug server communication interface operatively connected to the communication network 116 operatively connected to the administrative server 101 to provide the received hazardous drug changes to the hospital hazardous drug database 110a-110n, and/or a hospital server memory operatively connected to the hospital server 112a-112n for storing the received hazardous drug changes. In some embodiments, the administrative computing device of the administrative server 101 can determine if a received hazardous drug change is applicable to hazardous drug information stored on each of the hospital hazardous drug databases 110a-110n, and if so, the administrative server 101 can trigger hazardous drug update at respective hospital hazard drug databases 110a-110n or the hospital server memory of the hospital server 112a-112n by communicating over the communication network 116.

In some embodiments, the other hazardous drug database 108 can be any suitable database for storing data regarding hazardous drugs at a non-governmental entity or related to non-governmental rules for hazardous drugs. Examples of non-governmental agencies could include professional (doctor) groups, hazardous drug watch groups, etc. For example, the other hazardous drug database 108 can be a database that stores any suitable data, such as hazardous drug data, user data, client drug data, client drug selectable data, changelog data, client data, selectable data, safety data sheet (SDS) data, IP address data, file data, multum drug data, and/or any other suitable data. The administrative server 101 and the hospital servers 112a-112n can communicate with the other hazardous drug database 108 via a communication network 116. The storage of the other hazardous drug data, user data, client drug data, client drug selectable data, changelog data, client data, selectable data, safety data sheet (SDS) data, IP address data, file data, multum drug data and other information, programs, data and/or other suitable information on the administrative server 101, the other hazardous drug database, and the hospital hazardous drug databases 110a-110n can be implemented as digital data in any digital data format. In some embodiments, the other hazardous drug database 108 can include an other hazardous drug server computing device (e.g., hardware 200) having an other hazardous drug server communication interface operatively connected to the communication network 116 to receive hazardous drug updates from a hazardous drug manufacturer server 120 and operatively connected to the administrative server 101 to provide the received hazardous drug updates to the hospital hazardous drug database 110a-110n, and/or a hospital server memory operatively connected to the hospital server 112a-112n for storing the received hazardous drug updates. In some embodiments, the administrative computing device of the administrative server 101 can determine if a received hazardous drug update is applicable to a hazardous drug information stored on each of the hospital hazardous drug databases 110a-110n, and if so, the administrative server 101 can trigger a hazardous drug update at respective hospital hazard drug databases 110a-110n or the hospital server memory of the hospital server 112a-112n by communicating over the communication network 116.

In some embodiments, the other hazardous drug database 108 can include an other hazardous drug server computing device (e.g., hardware 200) having an other hazardous drug server input/output interface to receive hazardous drug changes from an other official with authorization to change the other hazardous drug data and an other hazardous drug server communication interface operatively connected to the communication network 116 operatively connected to the administrative server 101 to provide the received hazardous drug changes to the hospital hazardous drug database 110a-110n, and/or a hospital server memory operatively connected to the hospital server 112a-112n for storing the received hazardous drug changes. In some embodiments, the administrative computing device of the administrative server 101 can determine if a received hazardous drug change is applicable to a hazardous drug information stored on each of the hospital hazardous drug databases 110a-110n, and if so, the administrative server 101 can trigger a hazardous drug update at respective hospital hazard drug databases 110a-110n or the hospital server memory of the hospital server 112a-112n by communicating over the communication network 116.

In some embodiments, hazardous drug manufacturer servers 120 can be any suitable server for providing any suitable data, such as hazardous drug data. Hazardous drug manufacturer servers 120 can also supply other forms of hazardous drug data including, but not limited to, user data, client drug data, client drug selectable data, changelog data, client data, selectable data, safety data sheet (SDS) data, IP address data, file data, multum drug data, and/or any other suitable data.

In some embodiments, the administrative server 101 can be any suitable server for certifying hazardous drug data. For example, the administrative server 101 can be a server that receives hazardous drug data from a federal hazardous drug database 102, a state hazardous drug database 104, a local hazardous drug database 106, another hazardous drug database 108, a hospital hazardous drug database 110a-110n, and a hazardous drug manufacturer server 120 via a communication network 116, and/or stores the hazardous drug data and/or determines whether the hazardous drug data is accurate. The storage of the hazardous drug data, and other information, programs, data and/or other suitable information on the administrative server 101 can be implemented as digital data in any digital data format. In some embodiments, the administrative server 101 can include an administrative server computing device (e.g., hardware 200) having an administrative server communication interface operatively connected to the communication network 116 to receive hazardous drug data from federal hazardous drug database 102, a state hazardous drug database 104, a local hazardous drug database 106, another hazardous drug database 108, a hospital hazardous drug database 110a-110n, and a hazardous drug manufacturer server 120 and operatively connected to the administrative server computing device to provide the received hazardous drug data to the hospital server computing device, and/or a hospital server memory operatively connected to the hospital server computing device for storing the received hazardous drug data. In some embodiments, the administrative server computing device of the administrative server 101 can generate data models, for example hazardous drug data model, user data model, client drug data model, client drug selectable data model, changelog data model, client data model, selectable data model, safety data sheet (SDS) data model, IP address data model, file data model, multum drug data model and the generated data model can be transmitted by the administrative server communication interface to another location on the communication network 116, such as the hospital hazardous drug database 110a-110n or hospital server memory of the hospital server 112a-112n. In some embodiments, the administrative server computing device of the certification server 306 can generate a hazardous drug report based on the hazardous drug data and the hazardous drug data model, the user data and the user data model, the client drug data and the client drug data model, the client drug selectable data and the client drug selectable model, the changelog data and the changelog data model, the client data and the client data model, the selectable data and the selectable data model, the safety data sheet (SDS) data and the SDS data model, the IP address data and the IP address data model, the file data and the file data model, the multum drug data and the multum drug data model, and the hazardous drug report can be transmitted by the administrative server communication interface to another location on the communication network 116, such as the hospital hazardous drug database 110a-110n or hospital server memory of the hospital server 112a-112n.

Although the administrative server 101 and the hospital servers 112a-112n are illustrated as separate devices in FIG. 1, the functions performed by the administrative server 101 and the hospital servers 112a-112n can be performed using any suitable number of devices in some embodiments. For example, in some embodiments, the functions performed by either the administrative server 101 or the hospital servers 112a-112n can be performed on a single device. As another example, in some embodiments, multiple devices can be used to implement the functions performed by the administrative server 101 and the hospital servers 112a-112n.

The administrative server 101, the federal hazardous drug database 1-2, the state hazardous drug database 104, the local hazardous drug database 106, the other hazardous drug database 108, the hospital hazardous drug databases 110a-110n, the hospital servers 112a-112n, the user devices 114a-114n, and the hazardous drug manufacturer server 120 can be implemented using any suitable hardware in some embodiments. For example, in some embodiments, the administrative server 101, the federal hazardous drug database 1-2, the state hazardous drug database 104, the local hazardous drug database 106, the other hazardous drug database 108, the hospital hazardous drug databases 110a-110n, the hospital servers 112a-112n, the user devices 114a-114n, and the hazardous drug manufacturer server 120 can be implemented using any suitable general purpose computer or special purpose computer. Any such general purpose computer or special purpose computer can include any suitable hardware. Such hardware can include a hardware processor, a memory and/or storage, an input device controller, an input device, display/audio drivers, display and audio output circuitry, a communication interface(s), an antenna and a bus as further described herein.

Although FIG. 1 illustrates an example system 100 for administering safety information for hazardous drugs in accordance with this disclosure, various changes may be made to FIG. 1. For example, the number and placement of various components of the system 100 can vary as needed or desired. In addition, the system 100 may be used in any other suitable hazardous dug safety information administering process and is not limited to the specific processes described above.

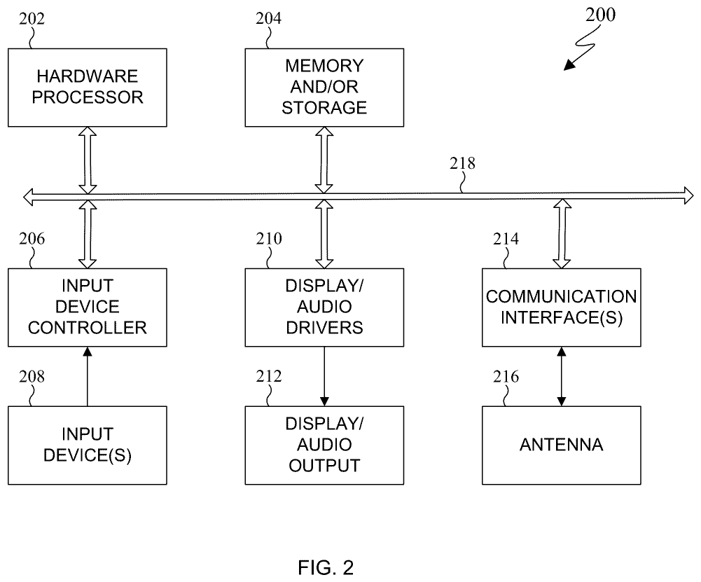

FIG. 2 illustrates an example system of hardware implemented as a computing device 200 for administering hazardous drug records in accordance with this disclosure. The embodiment of the computing device 200 illustrated in FIG. 2 is for illustration only. FIG. 2 does not limit the scope of this disclosure to any particular implementation of a computing device.

As shown in FIG. 2, there is illustrated one example of computer hardware 200 implemented as the administrative server 101, the federal hazardous drug database 1-2, the state hazardous drug database 104, the local hazardous drug database 106, the other hazardous drug database 108, the hospital hazardous drug databases 110a-110n, the hospital servers 112a-112n, the user devices 114a-114n, and the hazardous drug manufacturer server 120, respectively, in accordance with respective embodiments. In some other embodiments, any suitable computing device can be used. As illustrated in FIG. 2, the computer hardware 200 can include a hardware processor 202, a memory and/or storage 204, an input device controller 206, an input device 208, display/audio drivers 210, display and audio output circuitry 212, a communication interface(s) 214, an antenna 216 and a bus 218.

In certain embodiments, the hardware processor 202 can include any suitable hardware processor, such as a microprocessor, a micro-controller, digital signal processor(s), dedicated logic, and/or any other suitable circuitry for controlling the functioning of a general purpose computer or a special purpose computer. In some embodiments, the hardware processor 202 can be controlled by a program stored in the memory and/or storage 204. For example, the program can cause the hardware processor 202 to perform the mechanisms and/or processes described herein for managing hazardous drug data, and/or perform any other suitable actions.

In certain embodiments, the memory and/or storage 204 can be any suitable memory and/or storage for storing application information, programs, data, and/or any other suitable information. For example, the memory and/or storage 204 can include random access memory (“RAM”), read-only memory (“ROM”), flash memory, hard disk storage, optical media and/or any other suitable memory. In certain embodiments, the memory and/or storage can store the hazardous drug data.

In certain embodiments, the input device controller 206 can be any suitable circuitry for controlling and receiving input from one or more input devices 208. For example, the input device controller 206 can be circuitry for receiving input from a touchscreen, from a keyboard, from a mouse, from one or more buttons, from a voice recognition circuit, from a microphone, from a camera, from an optical sensor, from an accelerometer, from a temperature sensor, from a near field sensor, and/or from any other type of input device 208. In certain embodiments, the input device controller 206 can be utilized to receive hazardous drug data from one or more input devices 208.

In certain embodiments, the display/audio drivers 210 can be any suitable circuitry and/or software for controlling and driving output to one or more display/audio output devices 212. For example, the display/audio drivers 210 can be circuitry for driving a touchscreen, a flat-panel display, a cathode ray tube display, a projector, a speaker or speakers and/or any other suitable display and/or presentation devices 212. In certain embodiments, the display/audio drivers 210 can output or display the hazardous drug data to the display/audio output device 202

In certain embodiments, the communication interface(s) 214 can be any suitable circuitry and/or software for interfacing with one or more communication networks, such as the communication network 116 shown in FIG. 1 and previously described. For example, the interface(s) 214 can include network interface card circuitry, wireless communication circuitry and/or any other suitable type of communication network circuitry. The communication interface(s) 214 can also include circuitry for interfacing with external devices including the storage device and/or the memory 204 for storing and/or retrieving hazardous drug data from the storage device and/or the memory. In some embodiments, the hazardous drug data can be stored in the storage device and/or the memory 204 as digital data and/or can be transmitted to, or received from, the communication network as digital data.

The antenna 216 can be any of one or more suitable antennas for wirelessly communicating with a communication network (e.g., the communication network 116 of FIG. 1 as previously described). In some embodiments, the antenna 216 can be internal to the hardware 200 or omitted.

The bus 218 can be any suitable mechanism for communicating between two or more components in some embodiments. The communication between the components of the computer device 200 along the data bus 218 can be implemented as digital data.

Although FIG. 2 illustrates a computer device 200, various changes may be made to FIG. 2. For example, the number and placement of various components of the computing device 200 can vary as needed or desired. Any other suitable components can be included in computing device 200 in accordance with some embodiments. In addition, the computing device 200 may be used in any other suitable hazardous drug safety information administering process and is not limited to the specific processes described above.

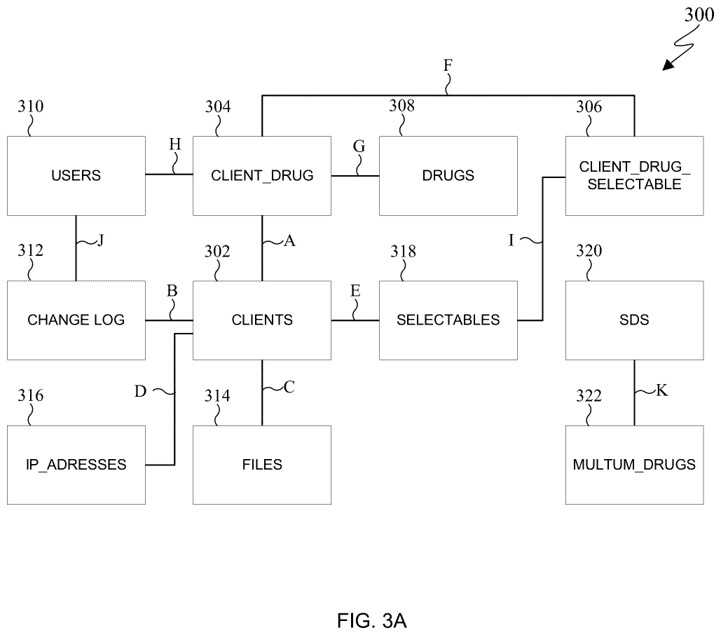

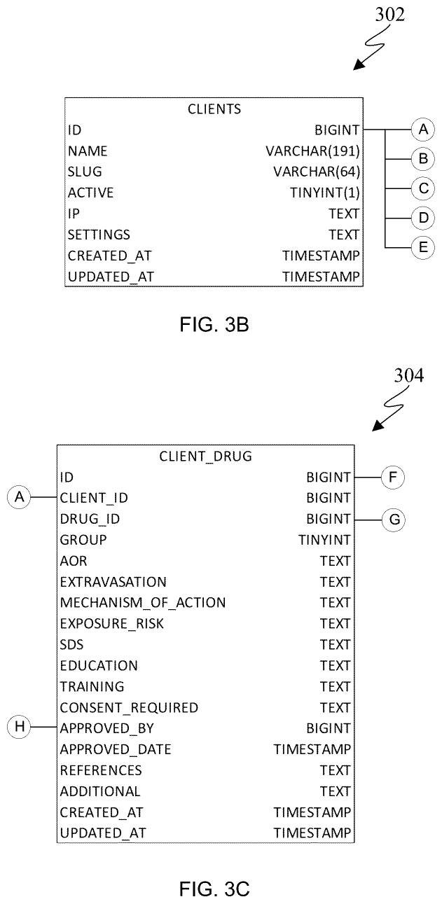

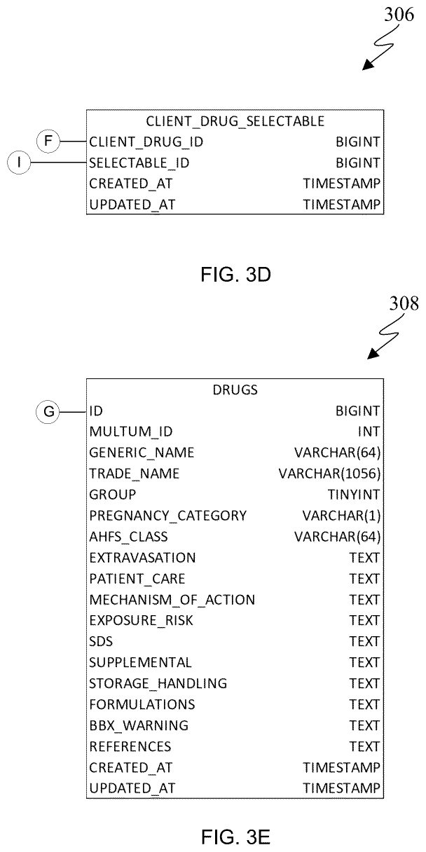

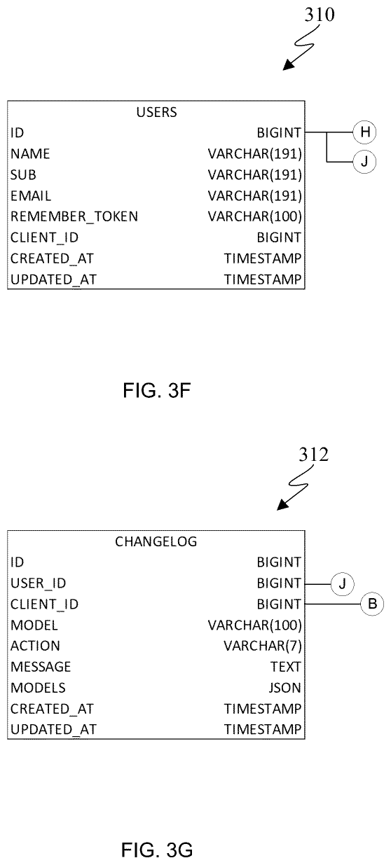

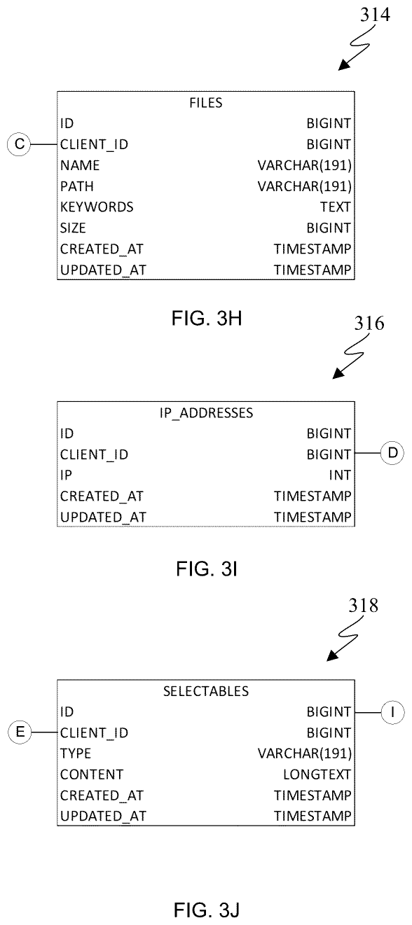

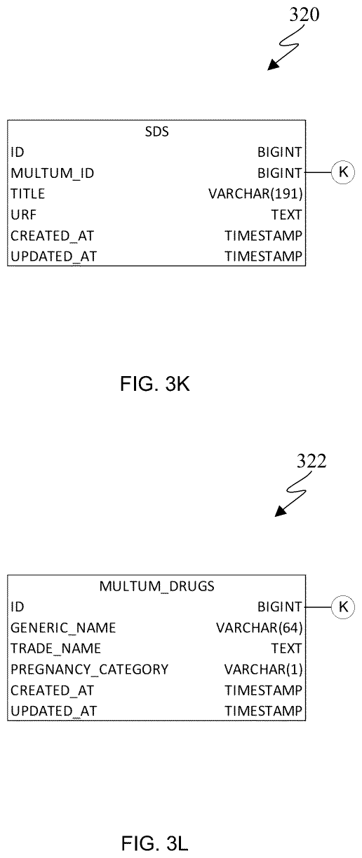

FIGS. 3A through 3L illustrate hazardous drug data structure 300 for a hazardous drug database in accordance with this disclosure. In particular, FIG. 3A illustrates overall hazardous drug data structure 300, FIG. 3B illustrates an example client data substructure 302, FIG. 3C illustrates an example client drug data substructure 304, FIG. 3D illustrates an example client drug selectable data substructure 306, FIG. 3E illustrates an example drugs data substructure 308, FIG. 3F illustrates an example users data substructure 310, FIG. 3G illustrates an example changelog data substructure 312, FIG. 3H illustrates an example files data substructure 314, FIG. 3I illustrates an example IP addresses data substructure 316, FIG. 3J illustrates an example selectables data substructure 318, FIG. 3K illustrates an example SDS data substructure 320, and FIG. 3L illustrates an example multum drugs data substructure 322. The embodiments of the hazardous drug data substructure 300 illustrated in FIGS. 3A through 3L are for illustration only. FIGS. 3A through 3L do not limit the scope of this disclosure to any particular implementation of a hazardous drug data structure.

As shown in FIG. 3A, a hazardous drug data structure 300 can store all the necessary hazardous drug data for optimal admiration. The hazardous drug data structure 300 can store all data related to hazardous drugs. In certain embodiments, the hazardous drug structure 300 can be used with the administrative server 101. The administrative server 101 can store all of the data related to all hazardous drug data from the federal hazardous drug databases 102, the state hazardous drug databases 104, the local hazardous drug databases 106, the other hazardous drug databases 108, the hospital hazardous drug databases 110a-110n, and the drug manufacturer databases 120.

In certain embodiments, hazardous drug data can be stored in multiple substructures 302-322 in the hazardous drug data structure 300. Examples of hazardous drug data stored in multiple places is indicated by the links A-K. When linked data is updated in one data substructure 302-322, each other instances of the linked data in other substructure are also updated.

In certain embodiments, the hazardous drug data structure 300 can be located on a hospital hazardous drug database 110 or a storage 204 of the hospital server 112. The hazardous drug data structure 300 can be customized based on the hospital hazardous drug database 110 or a storage 204 of the hospital server 112. For instance, a hospital may not want to keep IP address information and the IP address subsection 316 may not be utilized. As another example, a hospital may have different classifications of patients and require separate structures for client data substructures 302. Similarly, a hazardous drug data structure 300 could include different structures for user data substructures 310 for users with different levels or authorization to hazardous drugs.

The federal hazardous drug database 102, the state hazardous drug database 104, the local hazardous drug database 106, the other hazardous drug database 108, and the hazardous drug manufacturer server 120 may not use the hazardous drug data structure shown in FIG. 3A. In certain embodiments, the administrative server 101 can receive hazardous drug data in foreign data structures from each and any of federal hazardous drug database 102, the state hazardous drug database 104, the local hazardous drug database 106, the other hazardous drug database 108, and the hazardous drug manufacturer server 120. The administrative server 101 can identify source hazardous drug data in foreign data structures and incorporate the source hazardous drug data into the hazardous drug data structure 300.

As shown in FIG. 3B, the client data substructure 302 can contain hazardous drug data related to client data. Each client in a respective system can have a client data substructure 302. The client data substructure 302 can be shared with different hospital servers 112a-112n. In certain embodiments, the shared client data substructures 302 can be linked between hazardous drug data structures 300 at different hospitals. When the client data is updated at one hospital, then the linked client data substructures 302 at other hospitals are also updated. This ensures that each linked client data substructure 302 is updated.

In certain embodiments, when separate client data substructures 302 are created at different hospitals, the administrative server 101 can combine and link the client data into a single client data substructure 302 on the administrative server 101 that links the information at both hospitals. The administrative server 101 can update and insert client data that may be known to one hospital but unknown to other hospitals.

The administrative server 101 can also keep local copies of the hospital-specific client data substructures 302. For instance, when a hospital does not want to store specific information that may be stored at the administrative server 101 or other hospitals, a local copy of the client data substructure 302 can be stored at the administrative server 101.

Examples of client data can include a client ID field, a client name field, a client slug field, a client active field, a client IP field, a client settings field, a client created at field, a client updated at field, etc. In certain embodiments, the client data substructure 302 can be customized based on the requirements of a specified hospital. For example, a hospital may provide for privacy and associate a specific patient only with a patient ID field and not a client name field. In this case, someone that is not authorized to access the hazardous drug database would not be able to determine the client account based on a client name and have access to private data like drugs that the client is taking and the reasons or health issues related to the drugs.

The client ID field can include any identifying information of a client including a custom client ID, a driver's license, a social security number, an employee number, a license number, etc. The client ID field can be stored as a BIGINT. BIGINT is a special numeric type that provides support for integers of arbitrary length. As shown by links A-E, the client ID field can be linked to a client ID field in other substructures. Although the client ID field is the only linked field illustrated in FIG. 3B, any of the other fields can also be linked to corresponding fields in other substructures.

The client name field can be a name of a patient that corresponds to a respective client data substructure. The client name field can be stored as a VARCHAR(191). VARCHAR is a data type that store character strings of varying length that container single-byte and multibyte characters. The client name field can include a first name, a middle name, a nickname, a last name, etc.

The client slug field can be used as a unique identifier for a web address for a client. The client slug field can be stored as a VARCHAR(64). The client slug field can be an IP address, a random number provided in a separate table, a number generated using an algorithm, etc.

The client active field can indicate that the client is currently being prescribed a hazardous drug. The client active field can be stored as a TINYINT(1). TINYINT is a number type for a very small integer, typically in a range from −128 to 127. The client active field can be set to “0” or “1” for an active prescription and the opposite value for no active prescription.

The client IP field can be used to restrict access to a hazardous drug server. The client IP field can be stored as a text. The access to the hazardous drug server can be controlled by a network address of a specified client. After identifying a specific client data substructure, the administrative server can look up the client IP field to determine access to other data substructures within the hazardous drug data structure.

The client settings field can store customizable settings for a client hazardous drug substructure The client settings field can be stored as text. After identifying a specific client data substructure, the administrative server can look up the client settings field to determine client-specific settings for the hazardous drug data structure.

The client created at field can indicate a time and date at which the respective client subsection 302 was created. The client created at field can be stored as a timestamp. The client created at field can indicate a time that a client subsection 302 was created in general, was created at a specific hospital hazardous drug database 110, received from the administrative server 101, received from another hospital database 110a-110n, etc.

The client updated at field can indicate a time and date at which the respective client subsection 302 was last updated. The client updated at field can be stored as a timestamp. In certain embodiments, an additional client updated at field can be added for each time that the client subsection is updated. The additional client updated at fields can be ordered in an ascending or descending order based on the respective times.

As shown in FIG. 3C, the client drug data substructure 304 can contain hazardous drug data related to client drugs. Each client drug in a respective system can have a client drug data substructure 304. The client drug data substructure 304 can be shared with different hospital servers 112a-112n. In certain embodiments, the shared client drug data substructures 304 can be linked between hazardous drug data structures 300 at different hospitals. When the client drug data is updated at one hospital, then the linked client drug data substructures 304 at other hospitals are also updated. This ensures that each linked client drug data substructure 304 is updated.

In certain embodiments, when separate client drug data substructures 304 are created at different hospitals, the administrative server 101 can combine and link the client drug data into a single client drug data substructure 304 on the administrative server 101 that links the information at both hospitals. The administrative server 101 can update and insert client drug data that may be known to one hospital but unknown to other hospitals.

The administrative server 101 can also keep local copies of the hospital-specific client drug data substructures 304. For instance, when a hospital does not want to store specific information that may be stored at the administrative server 101 or other hospitals, a local copy of the client drug data substructure 304 can be stored at the administrative server 101.

Examples of client drug data can include a client drug ID field, a client drug client ID field, a client drug ID field, a client drug group field, a client drug AOR field, a client drug extravasation field, a client drug mechanism of action field, a client drug exposure risk field, a client drug safety data sheet (SDS) field, a client drug education field, a client drug training field, a client drug consent required field, a client drug approved by field, a client drug approved date field, a client drug references field, a client drug additional field, a client drug created at field, a client drug updated at field, etc. In certain embodiments, the client drug data substructure 304 can be customized based on the requirements of a specified hospital. For example, a hospital may combine the client drug references field and the client drug additional field for simplicity and include references in the client drug additional field.

The client drug ID field can include any identifying information of the client drug. For example, the client drug ID field can be given a unique number for the client drug data substructure 304. The client drug ID field can be stored as a BIGINT. As shown by link F, the client drug ID field can be linked to an ID field in other substructures.

The client drug client ID field can include any identifying information of a client including a custom client ID, a driver's license, a social security number, an employee number, a license number, etc. The client drug client ID field can be stored as a BIGINT. As shown by link A, the client drug client ID field can be linked to a client ID field in other substructures.

The client drug drug ID field can be an identification for a drug corresponding to the client. The client drug drug ID field can be stored as a BIGINT. As shown by link G, the client drug drug ID field can be linked to a drug ID field in other substructures.

The client drug group field can store a hazardous group classification. The client drug group field can be stored as a TINYINT. For example, a group designation from the National Institute for Occupational Safety and Health (NIOSH) is typically used for a client drug and an entry in the client drug group field can override the NIOSH group designation. The administrative server 101 can identify a custom hazardous group classification in the client drug group field and associate information for the client drug data substructure for each hazardous drug with a same value in the client drug group field.

The client drug AOR field can contain assessment of risk information for a drug for an institution of the client. The client drug AOR can be stored as text. The assessment of risk information can describe potential hazards or risk factors for different situations of handling a specific client drug.

The client drug extravasation field can contain extravasation information for the client drug data substructure. The client drug extravasation field can be stored as text. Extravasation is a leakage of fluid out of a container.

The client drug mechanism of action field can contain mechanism of action information. The client drug mechanism of action field can be stored as text. Mechanism of action information can include a specific interaction through which a hazardous drug produces paralogical effects. The mechanism of action information can include a specific molecular target for a client drug.

The client drug exposure risk field can contain exposure risk information. The client drug exposure risk field can be stored as text. Exposure risk information can include information for estimating or measuring a magnitude, frequency, and duration of exposure to a hazardous drug. The exposure risk information can also include hazard identification information and dose-response information.

The client drug SDS field can contain SDS links. The client drug SDS field can be stored as text. The SDS links can link a client drug data substructure to SDS data substructures. The client drug education field can contain education information. The client drug education field can be stored as text. The client drug training field can contain training information. The client drug training field can be stored as text.

The client drug consent required field can contain information on whether a hazardous drug requires approval to be used. The client drug consent required field can be stored as text. The client drug consent can include specific consent information to be provided to a client approving the hazardous drug. Examples of consent information can include warnings, possible side effects, etc.

The client drug approved by field can contain a user ID of a user who approved the information stored in the client drug data substructure. The client drug approved by field can be stored as a BIGINT. As shown by link H, the client drug approved be field can be linked to an approved by field in other substructures. The client drug approved date field can contain a date the information in the client drug data substructure was approved. The client drug approved date field can be stored as a timestamp.

The client drug references field can contain references for a hazardous drug. The client drug references field can be stored as text. The references can include medical studies, journal articles, etc. The references can be linked to the medical studies, journal articles, etc.

The client drug additional field can contain any additional information for a hazardous drug. The client drug additional field can be stored as text. The additional information can include information that exceeds a character limit in other client drug fields, helpful information that may not fit into a category or description for a client drug field, etc.

The client drug created at field can indicate a time and date at which the respective client drug subsection 302 was created. The client drug created at field can be stored as a timestamp. The client drug created at field can indicate a time that a client drug subsection 302 was created in general, was created at a specific hospital hazardous drug database 110, received from the administrative server 101, received from another hospital database 110a-110n, etc.

The client drug updated at field can indicate a time and date at which the respective client drug subsection 302 was last updated. The client drug updated at field can be stored as a timestamp. In certain embodiments, an additional client drug updated at field can be added for each time that the client drug subsection is updated. The additional client drug updated at fields can be ordered in an ascending or descending order based on the respective times.

As shown in FIG. 3D, the client drug selectable data substructure 306 can contain hazardous drug data related to various client drug selectables. Each client drug selectable in a respective system can have a client drug selectable data substructure 306. The client drug selectable data substructure 306 can be shared with different hospital servers 112a-112n. In certain embodiments, the shared client drug selectable data substructures 306 can be linked between hazardous drug data structures 300 at different hospitals. When the client drug selectable data is updated at one hospital, then the linked client drug selectable data substructures 306 at other hospitals are also updated. This ensures that each linked client drug selectable data substructure 306 is updated.

In certain embodiments, when separate client drug selectable data substructures 306 are created at different hospitals, the administrative server 101 can combine and link the client drug selectable data into a single client drug selectable data substructure 306 on the administrative server 101 that links the information at both hospitals. The administrative server 101 can update and insert client drug selectable data that may be known to one hospital but unknown to other hospitals.

The administrative server 101 can also keep local copies of the hospital-specific client drug selectable data substructures 306. For instance, when a hospital does not want to store specific information that may be stored at the administrative server 101 or other hospitals, a local copy of the client drug selectable data substructure 306 can be stored at the administrative server 101.

Examples of client drug selectable data can include a client drug selectable ID field, a client drug selectable selectable ID field, a client drug selectable created at field, a client drug selectable updated at field, etc. In certain embodiments, the client drug selectable data substructure 306 can be customized based on requirements of a specified hospital.

The client drug selectable client drug ID field can include any identifying information of the client drug. The client drug selectable ID field can be stored as a BIGINT. As shown by link F, the client drug selectable client drug ID field can be linked to a client drug ID field in other substructures.

The client drug selectable selectable ID field can contain a foreign key for a selectable paired with a given client drug. The client drug selectable selectable ID field can be stored as a BIGINT. As shown by link I, the client drug selectable selectable ID field can be linked to a selectable ID field in other substructures.

The client drug selectable created at field can indicate a time and date at which the respective client drug selectable subsection 302 was created. The client drug selectable created at field can be stored as a timestamp. The client drug selectable created at field can indicate a time that a client drug selectable subsection 302 was created in general, was created at a specific hospital hazardous drug database 110, received from the administrative server 101, received from another hospital database 110a-110n, etc.

The client drug selectable updated at field can indicate a time and date at which the respective client drug selectable subsection 302 was last updated. The client drug selectable updated at field can be stored as a timestamp. In certain embodiments, an additional client drug selectable updated at field can be added for each time that the client drug selectable subsection is updated. The additional client drug selectable updated at fields can be ordered in an ascending or descending order based on the respective times.

As shown in FIG. 3E, the drugs data substructure 308 can contain hazardous drug data related to drugs. Each drug in a respective system can have a drugs data substructure 308. The drugs data substructure 308 can be shared with different hospital servers 112a-112n. In certain embodiments, the shared drugs data substructures 308 can be linked between hazardous drug data structures 300 at different hospitals. When the drugs data is updated at one hospital, then the linked drugs data substructures 308 at other hospitals are also updated. This ensures that each linked drugs data substructure 308 is updated.

In certain embodiments, when separate drugs data substructures 308 are created at different hospitals, the administrative server 101 can combine and link the drugs data into a single drugs data substructure 308 on the administrative server 101 that links the information at both hospitals. The administrative server 101 can update and insert drugs data that may be known to one hospital but unknown to other hospitals.

The administrative server 101 can also keep local copies of the hospital-specific drugs data substructures 308. For instance, when a hospital does not want to store specific information that may be stored at the administrative server 101 or other hospitals, a local copy of the drugs data substructure 308 can be stored at the administrative server 101.

Examples of drug data can include a drugs ID field, a drugs multum ID field, a drugs generic name field, a drugs trade name field, a drugs group field, a drugs pregnancy category field, a drugs American Hospital Formulary Service (AHFS) class field, a drugs extravasation field, a drugs patient care field, a drugs mechanism of action field, a drugs exposure risk field, a drugs SPS field, a drugs supplemental field, a drugs storage handling field, a drugs formulations field, a drugs black box (BBX) warning field, a drugs reference field, a drugs created at field, a drug updated at field, etc. In certain embodiments, the drugs data substructure 308 can be customized based on requirements of a specified hospital.

The drugs ID field can contain a primary key for each drug in a table. The drugs ID field can be stored as a BIGINT. As shown by link G, the drugs ID field can be linked to an ID field in other substructures. The primary key is designated to uniquely identify each table record.

The drugs multum ID field can contain a foreign key identifying an item from the multum drugs table. The drugs multum ID field can be stored as an INT.

The drugs generic name field can contain a generic name of a hazardous drug for the drugs data substructure. The drugs generic name field can be stored as a VARCHAR(64). A non-limiting example of a generic name is abacavir.

The drugs trade name field can contain a brand or trade name for a hazardous drug for the drugs data substructure. The drugs trade name field can be stored as a VARCHAR(1056). A non-limiting example of a brand or trade name is abacavir sulfate or Ziagen.

The drugs group field can contain a hazardous group name approved by NIOSH. The drugs group field can be stored as a TINYINT. Examples of groups can include 1, 2, and 3. The group names are standardized by NIOSH.

The drugs pregnancy category field can contain a pregnancy category designation. The drugs pregnancy category field can be stored as a VARCHAR(1). The pregnancy category designation can be as A, B, C, D, X, N, etc. A: Adequate and well-controlled studies have failed to demonstrate a risk to the fetus in the first trimester of pregnancy (and there is no evidence of risk in later trimesters). B: Animal reproduction studies have failed to demonstrate a risk to the fetus and there are no adequate and well-controlled studies in pregnant women. C: Animal reproduction studies have shown an adverse effect on the fetus and there are no adequate and well-controlled studies in humans, but potential benefits may warrant use of the drug in pregnant women despite potential risks. D: There is positive evidence of human fetal risk based on adverse reaction data from investigational or marketing experience or studies in humans, but potential benefits may warrant use of the drug in pregnant women despite potential risks. X: Studies in animals or humans have demonstrated fetal abnormalities and/or there is positive evidence of human fetal risk based on adverse reaction data from investigational or marketing experience, and the risks involved in use of the drug in pregnant women clearly outweigh potential benefits. N: No category assigned.

The drugs AHFS class field can contain an AHFS therapeutic class for a hazardous drug. The drugs AHFS class field can be stored as a VARCHAR(64). The AHFS therapeutic class is based on a national system that acts as a logical way to group drugs for easy comparison as well as aggregate reporting on drugs for utilization and billing.

The drugs extravasation field can contain extravasation information for a hazardous drug. The drugs extravasation field can be stored as a text. Extravasation information can include information related to the hazardous drug leaking from a container.

The drugs patient care field can contain specific patient care information for a hazardous drug. The drugs patient care field can be stored as a text. The patient care information can include the amount of hazardous drug used, other procedures used for the application of the hazardous drug, etc.

The drugs mechanism of action field can contain mechanism of action information for a hazardous drug. The drugs mechanism of action field can be stored as a text. The mechanism of action information can describe how a hazardous drug produces an effect in the body. Mechanisms of action can include how a hazardous drug affects a specific target in a cell or a cell function. Examples of mechanisms of action can include second-messenger mechanisms, direct gene activation, inhibition of bacterial protein synthesis, inhibition of cell wall synthesis, inhibition of enzymatic activity, alteration of cell membrane permeability, and blockade of specific biochemical pathways.

The drugs exposure risk field can contain exposure risk information for a hazardous drug. The drugs exposure risk field can be stored as a text. The exposure risk information can be defined by a chance of harmful effects to human health or the ecological systems resulting from exposure to a hazardous drug.

The drugs SDS field can contain safety data sheet links for a hazardous drug. The drugs SDS field can be stored as a text. The SDS links can link a drugs data substructure to SDS data substructures.

The drugs supplemental field can contain supplemental information for a hazardous drug. The drugs supplemental field can be stored as a text.

The drugs storage handling field can contain storage and handling requirements for a hazardous drug. The drugs storage handling field can be stored as a text. Examples of storage and handling requirements can include required safety gear, safety procedures, etc. for storing and handling the hazardous drug.

The drugs formulations field can contain information about available formulations for a hazardous drug. The drugs formulations field can be stored as a text. The drug formulation can include multistep processes where an active drug is mixed with other components. The drug formulation information can also include dosage forms for a hazardous drug product as marketed using a specific mixture of active and inactive components.

The drugs BBX warning field can contain one or more links to an external server for accessing boxed warnings for a hazardous drug. The drugs BBX warning field can be stored as a text. Black box warnings can be required by the U.S. Food and Drug Administration for certain hazardous drugs. The black box warning can communicate potentially dangerous side effects or important instructions for safe use of a hazardous drug.

The drugs reference field can contain additional reference information or links for a hazardous drug. The drugs reference field can be stored as text.

The drug created at field can indicate a time and date at which the respective drug subsection 302 was created. The drug created at field can be stored as a timestamp. The drug created at field can indicate a time that a drug subsection 302 was created in general, was created at a specific hospital hazardous drug database 110, received from the administrative server 101, received from another hospital database 110a-110n, etc.

The drug updated at field can indicate a time and date at which the respective drug subsection 302 was last updated. The drug updated at field can be stored as a timestamp. In certain embodiments, an additional drug updated at field can be added for each time that the drug subsection is updated. The additional drug updated at fields can be ordered in an ascending or descending order based on the respective times.

As shown in FIG. 3F, the users data substructure 310 can contain hazardous drug data related to users. Each users in a respective system can have a users data substructure 310. The users data substructure 310 can be shared with different hospital servers 112a-112n. In certain embodiments, the shared users data substructures 310 can be linked between hazardous drug data structures 300 at different hospitals. When the users data is updated at one hospital, then the linked users data substructures 310 at other hospitals are also updated. This ensures that each linked users data substructure 310 is updated.

In certain embodiments, when separate users data substructures 310 are created at different hospitals, the administrative server 101 can combine and link the users data into a single users data substructure 310 on the administrative server 101 that links the information at both hospitals. The administrative server 101 can update and insert users data that may be known to one hospital but unknown to other hospitals.

The administrative server 101 can also keep local copies of the hospital-specific users data substructures 310. For instance, when a hospital does not want to store specific information that may be stored at the administrative server 101 or other hospitals, a local copy of the users data substructure 310 can be stored at the administrative server 101.

Examples of users data can include a users ID field, a users name field, a users sub field, a users email field, a users remember token field, a users client ID field, a users created at field, a users updated at field, etc. In certain embodiments, the users data substructure 310 can be customized based on requirements of a specified hospital.

The users ID field can include a primary key for a users table. The users ID field can be stored as text. As shown by links H and J, the user ID field can be linked to an ID field in other substructures.

The users name field can contain a name of a user. The users name field can be stored as text. The name of the user can include a first name, middle name, nickname, and last name.

The users sub field can include an identifier used to authenticate a user with an authentication provider. The users sub field can be stored as text.

The users email field can include an email address of the user. The users email field can be stored as text. The administrative server can use the email address to send hazardous drug information to a specific user.

The users remember token field can contain a unique token corresponding to a user. The users remember token field can be stored as text. The unique token can be stored on a user equipment of the user. The unique token can be used by the administrative server to identify a user from a previous login.

The users client ID field can contain a foreign key for a client table. The users client ID field can be stored as text. The foreign key can be used with the client table for linking a user to a single client.

The users created at field can indicate a time and date at which the respective users subsection 302 was created. The users created at field can be stored as a timestamp. The users created at field can indicate a time that a users subsection 302 was created in general, was created at a specific hospital hazardous drug database 110, received from the administrative server 101, received from another hospital database 110a-110n, etc.

The users updated at field can indicate a time and date at which the respective users subsection 302 was last updated. The users updated at field can be stored as a timestamp. In certain embodiments, an additional users updated at field can be added for each time that the users subsection is updated. The additional users updated at fields can be ordered in an ascending or descending order based on the respective times.

As shown in FIG. 3G, the changelog data substructure 312 can contain hazardous drug data related to changelogs. Each changelog in a respective system can have a changelog data substructure 312. The changelog data substructure 312 can be shared with different hospital servers 112a-112n. In certain embodiments, the shared changelog data substructures 312 can be linked between hazardous drug data structures 300 at different hospitals. When the changelog data is updated at one hospital, then the linked changelog data substructures 312 at other hospitals are also updated. This ensures that each linked changelog data substructure 312 is updated.

In certain embodiments, when separate changelog data substructures 312 are created at different hospitals, the administrative server 101 can combine and link the changelog data into a single changelog data substructure 312 on the administrative server 101 that links the information at both hospitals. The administrative server 101 can update and insert changelog data that may be known to one hospital but unknown to other hospitals.

The administrative server 101 can also keep local copies of the hospital-specific changelog data substructures 312. For instance, when a hospital does not want to store specific information that may be stored at the administrative server 101 or other hospitals, a local copy of the changelog data substructure 312 can be stored at the administrative server 101.

Examples of changelog data can include a changelog ID field, a changelog user ID field, a changelog client ID field, a changelog model field, a changelog action field, a changelog message field, a changelog models field, a changelog created at field, a changelog updated at field, etc. In certain embodiments, the changelog data substructure 312 can be customized based on requirements of a specified hospital.

The changelog ID field can contain a primary key. The users client ID field can be stored as text.

The changelog user ID field can contain a foreign key for the users table identifying the user that initiated a specified data update. The users client ID field can be stored as text. As shown by link J, the changelog user ID field can be linked to a user ID field in other substructures.

The changelog client ID field can contain a foreign key for a client table. The users client ID field can be stored as text. As shown by link B, the changelog client ID field can be linked to a client ID field in other substructures. The foreign key for the clients table can identify a client whose data was changed in any given data update.

The changelog model field can contain an identifier that indicates what type of data was changed in any given update. The users client ID field can be stored as text.

The changelog action field can contain an identifier that indicates an action completed in a data update. The users client ID field can be stored as text. Examples of a data update can include “created,” “updated,” “deleted,” etc.

The changelog message field can contain JSON encoded information that contains data that was saved in a data update. The users client ID field can be stored as text. While Java example are used, the description is not limited to using Java.

The changelog models field can contain JSON encoded data on both a previous and updated version of data in a data update. The users client ID field can be stored as text.

The changelog created at field can indicate a time and date at which the respective changelog subsection 302 was created. The changelog created at field can be stored as a timestamp. The changelog created at field can indicate a time that a changelog subsection 302 was created in general, was created at a specific hospital hazardous drug database 110, received from the administrative server 101, received from another hospital database 110a-110n, etc.

The changelog updated at field can indicate a time and date at which the respective changelog subsection 302 was last updated. The changelog updated at field can be stored as a timestamp. In certain embodiments, an additional changelog updated at field can be added for each time that the changelog subsection is updated. The additional changelog updated at fields can be ordered in an ascending or descending order based on the respective times.

As shown in FIG. 3H, the files data substructure 314 can contain hazardous drug data related to files. Each files in a respective system can have a files data substructure 314. The files data substructure 314 can be shared with different hospital servers 112a-112n. In certain embodiments, the shared files data substructures 314 can be linked between hazardous drug data structures 300 at different hospitals. When the files data is updated at one hospital, then the linked files data substructures 314 at other hospitals are also updated. This ensures that each linked files data substructure 314 is updated.

In certain embodiments, when separate files data substructures 314 are created at different hospitals, the administrative server 101 can combine and link the files data into a single files data substructure 314 on the administrative server 101 that links the information at both hospitals. The administrative server 101 can update and insert files data that may be known to one hospital but unknown to other hospitals.

The administrative server 101 can also keep local copies of the hospital-specific files data substructures 314. For instance, when a hospital does not want to store specific information that may be stored at the administrative server 101 or other hospitals, a local copy of the files data substructure 314 can be stored at the administrative server 101.

Examples of files data can include a files ID field, a files client ID field, a files name field, a files path field, a files keywords field, a files size field, a files created at field, a files updated at field, etc. In certain embodiments, the files data substructure 314 can be customized based on requirements of a specified hospital.

The files ID field can contain a primary key for the files table. The files ID field can be stored as BIGINT.

The files client ID field can be an ID of a client for the files substructure. The files client ID field can be stored as BIGINT. As shown by link C, the files client ID field can be linked to a client ID field in other substructures.

The files name field can contain a file name. The files name field can be stored as VARCHAR(191).

The files path field can contain a location of a file on a file system of a hazardous drug server. The files path field can be stored as VARCHAR(191). The files path field can be a location on an administrative server and a hospital server.

The files keywords field can contain searchable keywords for a file. The files keywords field can be stored as text. The keywords can be automatically generated or customizable based on how a specific user remembers a specific hazardous drug.

The files size field can contain information indicating a size of a file. The files size field can be stored as BIGINT. The size of the file can be based on an amount of bytes for a file.

The files created at field can indicate a time and date at which the respective files subsection 302 was created. The files created at field can be stored as a timestamp. The files created at field can indicate a time that a files subsection 302 was created in general, was created at a specific hospital hazardous drug database 110, received from the administrative server 101, received from another hospital database 110a-110n, etc.

The files updated at field can indicate a time and date at which the respective files subsection 302 was last updated. The files updated at field can be stored as a timestamp. In certain embodiments, an additional files updated at field can be added for each time that the files subsection is updated. The additional files updated at fields can be ordered in an ascending or descending order based on the respective times.

As shown in FIG. 3I, the IP addresses data substructure 316 can contain hazardous drug data related to IP addresses. Each IP addresses in a respective system can have an IP addresses data substructure 316. The IP addresses data substructure 316 can be shared with different hospital server 112a-112n. In certain embodiments, the shared IP addresses data substructures 316 can be linked between hazardous drug data structures 300 at different hospitals. When the IP addresses data is updated at one hospital, then the linked IP addresses data substructures 316 at other hospitals are also updated. This ensures that each linked IP addresses data substructure 316 is updated.

In certain embodiments, when separate IP addresses data substructures 316 are created at different hospitals, the administrative server 101 can combine and link the IP addresses data into a single IP addresses data substructure 316 on the administrative server 101 that links the information at both hospitals. The administrative server 101 can update and insert IP addresses data that may be known to one hospital but unknown to other hospitals.

The administrative server 101 can also keep local copies of the hospital-specific IP addresses data substructures 316. For instance, when a hospital does not want to store specific information that may be stored at the administrative server 101 or other hospitals, a local copy of the IP addresses data substructure 316 can be stored at the administrative server 101.

Examples of IP addresses data can include an IP addresses ID field, an IP addresses client ID field, an IP addresses IP field, an IP addresses created at field, an IP addresses updated at field, etc. In certain embodiments, the IP addresses data substructure 316 can be customized based on requirements of a specified hospital.

The IP addresses ID field can contain a primary key for an IP addresses table. The files size field can be stored as BIGINT.

The IP addresses client ID field can contain a foreign key to a clients table linking an IP address to a client. The files size field can be stored as BIGINT. As shown by link D, the IP addresses client ID field can be linked to a client ID field in other substructures.

The IP addresses IP field can contain an IP address that is allowed access to a hospital specific data structure stored at the administrative server or administrative database. The files size field can be stored as BIGINT. The administrative server can compare the IP addresses IP field to allow access to a hospital specific data structure.

The IP addresses created at field can indicate a time and date at which the respective IP addresses subsection 302 was created. The IP addresses created at field can be stored as a timestamp. The IP addresses created at field can indicate a time that a IP addresses subsection 302 was created in general, was created at a specific hospital hazardous drug database 110, received from the administrative server 101, received from another hospital database 110a-110n, etc.

The IP addresses updated at field can indicate a time and date at which the respective IP addresses subsection 302 was last updated. The IP addresses updated at field can be stored as a timestamp. In certain embodiments, an additional IP addresses updated at field can be added for each time that the IP addresses subsection is updated. The additional IP addresses updated at fields can be ordered in an ascending or descending order based on the respective times.

As shown in FIG. 3J, the selectables data substructure 318 can contain hazardous drug data related to selectables. Each selectables in a respective system can have a selectables data substructure 318. The selectables data substructure 318 can be shared with different hospital servers 112a-112n. In certain embodiments, the shared selectables data substructures 318 can be linked between hazardous drug data structures 300 at different hospitals. When the selectables data is updated at one hospital, then the linked selectables data substructures 318 at other hospitals are also updated. This ensures that each linked selectables data substructure 318 is updated.

In certain embodiments, when separate selectables data substructures 318 are created at different hospitals, the administrative server 101 can combine and link the selectables data into a single selectables data substructure 318 on the administrative server 101 that links the information at both hospitals. The administrative server 101 can update and insert selectables data that may be known to one hospital but unknown to other hospitals.

The administrative server 101 can also keep local copies of the hospital-specific selectables data substructures 318. For instance, when a hospital does not want to store specific information that may be stored at the administrative server 101 or other hospitals, a local copy of the selectables data substructure 318 can be stored at the administrative server 101.

Examples of selectables data can include a selectables ID field, a selectables client ID field, a selectables type field, a selectables content field, a selectables created at field, a selectables updated at field, etc. In certain embodiments, the selectables data substructure 318 can be customized based on requirements of a specified hospital.

The selectables ID field can include a primary key for a selectables table. The selectables ID field can be stored as BIGINT. As shown by link I, the selectables ID field can be linked to an ID field in other substructures.

The selectables client ID field can include a foreign key for the client table linking a selectable to a client. The selectables client ID field can be stored as BIGINT. As shown by link E, the selectables client ID field can be linked to a client ID field in other substructures.

The selectables type field can include an identifier that designates a type of selectable. The selectables type field can be stored as VARCHAR(191). Examples of selectable can include “administration,” “patient care,” “disposal,” etc.

The selectables content field can include each selectables content. The selectables content field can be stored as LONGTEXT. LONGTEXT is a data object for use in extreme string storage use cases, sometime used for up to 4,294,967,295 characters.

The selectables created at field can indicate a time and date at which the respective selectables subsection 302 was created. The selectables created at field can be stored as a timestamp. The selectables created at field can indicate a time that a selectables subsection 302 was created in general, was created at a specific hospital hazardous drug database 110, received from the administrative server 101, received from another hospital database 110a-110n, etc.

The selectables updated at field can indicate a time and date at which the respective selectables subsection 302 was last updated. The selectables updated at field can be stored as a timestamp. In certain embodiments, an additional selectables updated at field can be added for each time that the selectables subsection is updated. The additional selectables updated at fields can be ordered in an ascending or descending order based on the respective times.

As shown in FIG. 3K, the SDS data substructure 320 can contain hazardous drug data related to SDS. Each SDS in a respective system can have a SDS data substructure 320. The SDS data substructure 320 can be shared with different hospital servers 112a-112n. In certain embodiments, the shared SDS data substructures 320 can be linked between hazardous drug data structures 300 at different hospitals. When the SDS data is updated at one hospital, then the linked SDS data substructures 320 at other hospitals are also updated. This ensures that each linked SDS data substructure 320 is updated.

In certain embodiments, when separate SDS data substructures 320 are created at different hospitals, the administrative server 101 can combine and link the SDS data into a single SDS data substructure 320 on the administrative server 101 that links the information at both hospitals. The administrative server 101 can update and insert SDS data that may be known to one hospital but unknown to other hospitals.

The administrative server 101 can also keep local copies of the hospital-specific SDS data substructures 320. For instance, when a hospital does not want to store specific information that may be stored at the administrative server 101 or other hospitals, a local copy of the SDS data substructure 320 can be stored at the administrative server 101.

Examples of SDS data can include a SDS ID field, a SDS multum ID field, a SDS title field, a SDS unit risk factor (URF) field, a SDS created at field, a SDS updated at field, etc. In certain embodiments, the SDS data substructure 320 can be customized based on requirements of a specified hospital.

The SDS ID field can include a primary key for an SDS table. The selectables content field can be stored as LONGTEXT.

The SDS multum ID field can include a foreign key linking an SDS entry to the multum drugs table. The SDS multum ID field can be stored as a BIGINT. As shown by link K, the SDS multum ID field can be linked to a multum ID field in other substructures.

The SDS title field can include a title for a given SDS entry. The SDS title field can be stored as LONGTEXT.

The SDS URL field can contain a web URL to a safety data sheet document. The SDS URL field can be stored as LONGTEXT.

The SDS created at field can indicate a time and date at which the respective SDS subsection 302 was created. The SDS created at field can be stored as a timestamp. The SDS created at field can indicate a time that a SDS subsection 302 was created in general, was created at a specific hospital hazardous drug database 110, received from the administrative server 101, received from another hospital database 110a-110n, etc.

The SDS updated at field can indicate a time and date at which the respective SDS subsection 302 was last updated. The SDS updated at field can be stored as a timestamp. In certain embodiments, an additional SDS updated at field can be added for each time that the SDS subsection is updated. The additional SDS updated at fields can be ordered in an ascending or descending order based on the respective times.

As shown in FIG. 3L, the multum drugs data substructure 322 can contain hazardous drug data related to multum drugs. Each multum drug in a respective system can have a multum drug data substructure 322. The multum drug data substructure 322 can be shared with different hospital server 112a-112n. In certain embodiments, the shared multum drug data substructures 322 can be linked between hazardous drug data structures 300 at different hospitals. When the multum drug data is updated at one hospital, then the linked multum drug data substructures 322 at other hospitals are also updated. This ensures that each linked multum drug data substructure 322 is updated.

In certain embodiments, when separate multum drug data substructures 322 are created at different hospitals, the administrative server 101 can combine and link the multum drug data into a single multum drug data substructure 322 on the administrative server 101 that links the information at both hospitals. The administrative server 101 can update and insert multum drug data that may be known to one hospital but unknown to other hospitals.

The administrative server 101 can also keep local copies of the hospital-specific multum drug data substructures 322. For instance, when a hospital does not want to store specific information that may be stored at the administrative server 101 or other hospitals, a local copy of the multum drug data substructure 322 can be stored at the administrative server 101.

Examples of multum drug data can include a multum drug ID field, a multum drug generic name field, a multum drug trade name field, a multum drug pregnancy category field, a multum drug created at field, a multum drug updated at field, etc. In certain embodiments, the multum drug data substructure 322 can be customized based on requirements of a specified hospital.

The multum drug ID field can contain a primary key for a multum drugs table. The multum drug ID field can be stored as BIGINT. As shown by link K, the multum drug ID field can be linked to an ID field in other substructures.

The multum drug generic name field can a generic name assigned by Multum. The multum drug generic name field can be stored as VARCHAR(64).

The multum drug trade name field can include a brand or trade name identified by Multum for a given drug. The multum drug trade name field can be stored as text.

The multum drug pregnancy category field can include a pregnancy category designation for a given drug if available. The multum drug pregnancy category field can be stored as VARCHAR(1).

The multum drug created at field can indicate a time and date at which the respective multum drug subsection 302 was created. The multum drug created at field can be stored as a timestamp. The multum drug created at field can indicate a time that a multum drug subsection 302 was created in general, was created at a specific hospital hazardous drug database 110, received from the administrative server 101, received from another hospital database 110a-110n, etc.

The multum drug updated at field can indicate a time and date at which the respective multum drug subsection 302 was last updated. The multum drug updated at field can be stored as a timestamp. In certain embodiments, an additional multum drug updated at field can be added for each time that the multum drug subsection is updated. The additional multum drug updated at fields can be ordered in an ascending or descending order based on the respective times.

Although FIGS. 3A through 3L illustrate a hazardous drug data structure 300, various changes may be made to FIGS. 3A through 3L. For example, the number and placement of various components of the hazardous drug data structure 300 can vary as needed or desired. In addition, the hazardous drug data structure 300 may be used in any other suitable hazardous drug administration process and is not limited to the specific processes described above.

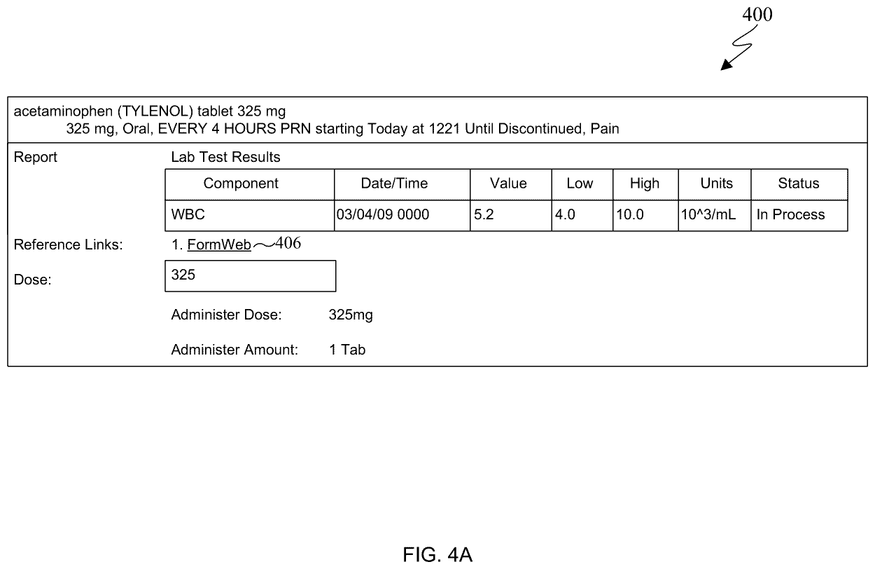

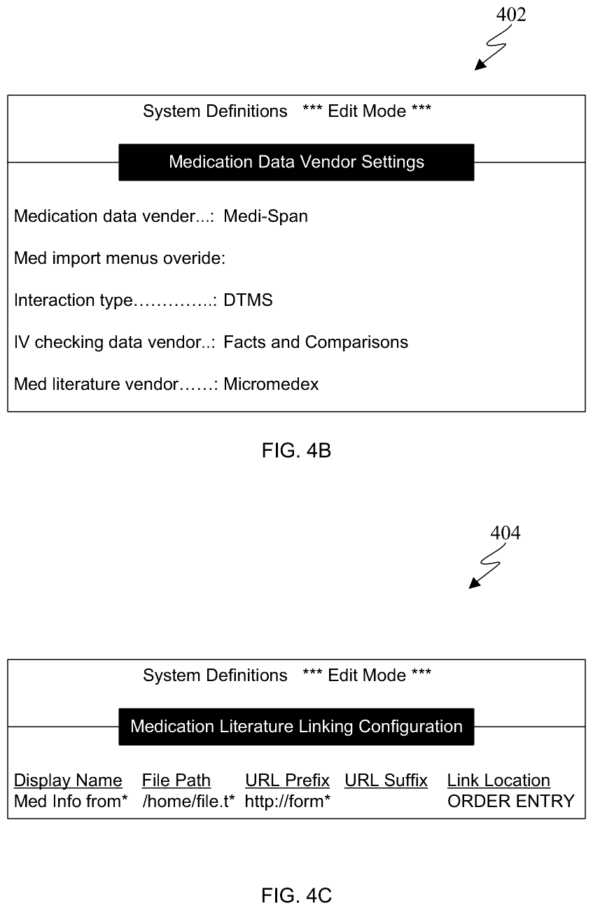

FIGS. 4A through 4C illustrate an example hazardous drug file setup in accordance with this disclosure. In particular, FIG. 4A illustrates hazardous drug record 400, FIG. 4B illustrates an example medication data vendor settings 402, and FIG. 4C illustrates an example medication literature linking configuration 404. The embodiments of the hazardous drug record 400, medication data vendor settings 402, medication literature linking configuration 404 illustrated in FIGS. 4A through 4C are for illustration only. FIGS. 4A through 4C do not limit the scope of this disclosure to any particular implementation of a hazardous drug data structure.

As shown in FIGS. 4A through 4C, reference links 406 are hyperlinks to medication information within an e-Prescribing (ERX) record that are embedded into the medication order entry activity or the MAR. A reference link 406 can be a display item and can be specified in an OCC record. It is stored in the master file ERX, item numbers 1310, 24130.

In order to set up a reference link 406, a pointer file can be obtained from the administrative server 101. As non-limiting examples, a pointer file can be named FWNDC.txt for FormWEb or RHNDC.txt for Rhazdrugs. The pointer file is maintained by the administrative server 101 and can contain the reference links 406 and corresponding NDCs.

Before running the utility to link ERX records to the reference links 406, settings need to be in place on the administrative server 101. As shown in FIG. 4B, an appropriate literature vendor should be entered in the Med literature vendor field of the system definitions in the medication data vendor settings 402.

As shown in FIG. 4C, a medication literature linking configuration 404 can be navigated to from a medications and allergens menu. The fields in the medication literature linking configuration 404 can be filled out to indicate a hazardous drug data structure. The display name field will represent a display name for the hyperlink in the hazardous drug record 400. The display name can appear in an order entry and MAR. The file path field is a path to the point file. The pointer file is the directory where the FWNDC.txt and RHNDC.txt files are stored. The URL prefix field and the URL suffix field can be locations of files on the administrative server or the hospital server. The prefix can vary based on the hazardous drug structure used. As a non-limiting example, the hazardous drug structure can be FormWeb, the web site URL can be http://formweb.com/kchc, and the URL prefix would be http://formweb.com/master/display.php?referrer=epic&dir=kchc&thedrug=. The value after dir=′ in the prefix should match a directory in a hospital hazardous drug data structure. As another non-limiting example, the hazardous drug structure can be Rhazdrugs, the website URL can be https://rhazdrugs.com/utsw, and the URL prefix would be https://rhazdrugs.com/utsw/drug/. The URL suffix can be left blank. The link location field can be MAR, order entry, or both.

The utility to link the ERX records with the reference files can be run once the settings are applied that are illustrated in FIGS. 4B and 4C. For example, a medication literature link RXLINK utility can be run from medication data vendor settings 402. The utility can remove all current medication reference links and add or update the medication reference links 406. The utility can allow designating locations for the reference links, select a vendor, and specify a full path to a hazardous drug data structure.

Although FIGS. 4A through 4C illustrate a hazardous drug file setup, various changes may be made to FIGS. 4A through 4C. For example, the number and placement of various components of the hazardous drug record 400, medication data vendor settings 402, medication literature linking configuration 404 can vary as needed or desired. In addition, the hazardous drug record 400, medication data vendor settings 402, medication literature linking configuration 404 may be used in any other suitable hazardous drug administrative process and is not limited to the specific processes described above.

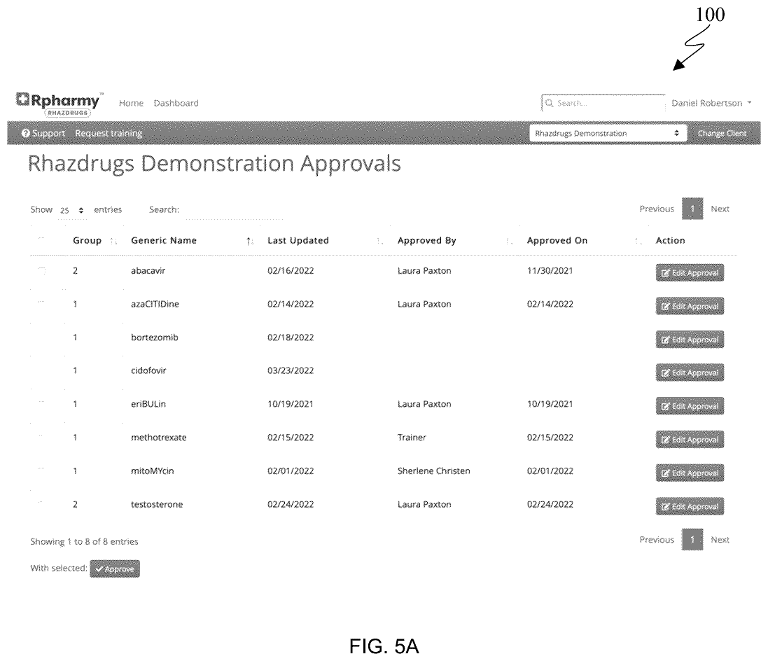

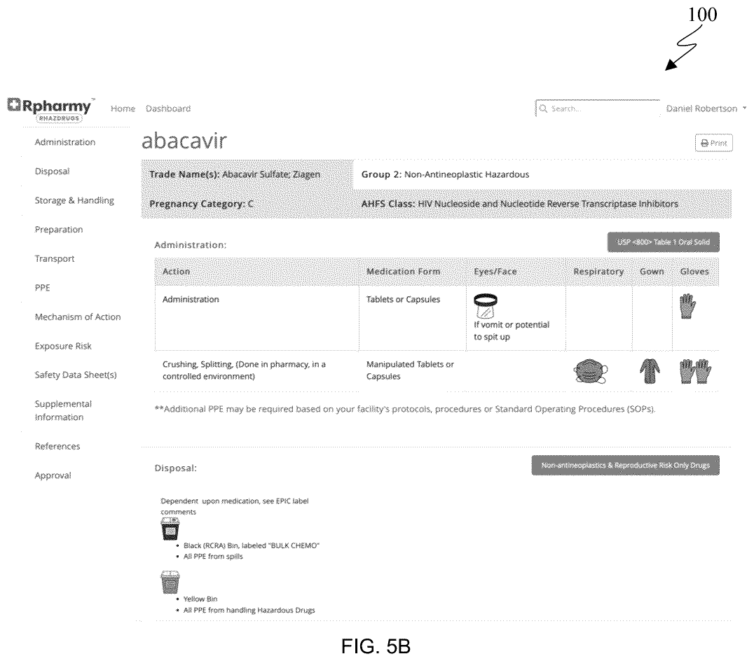

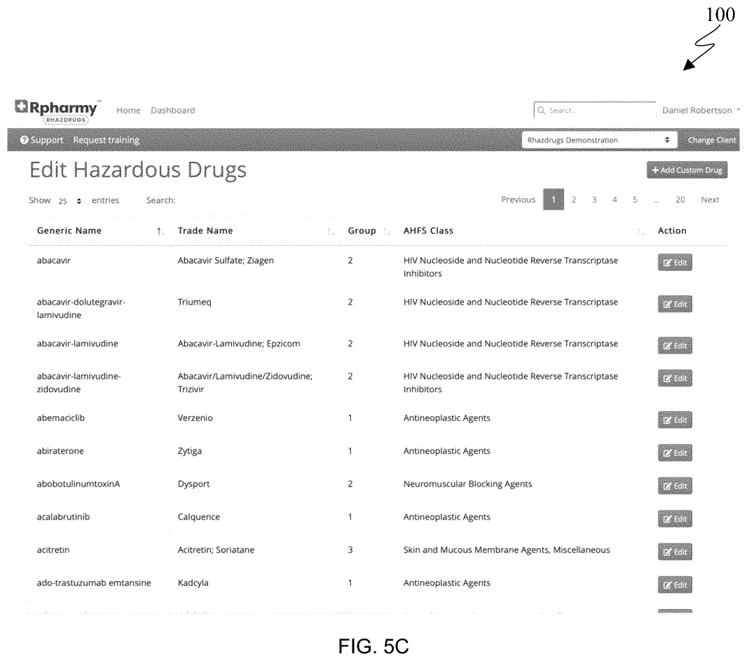

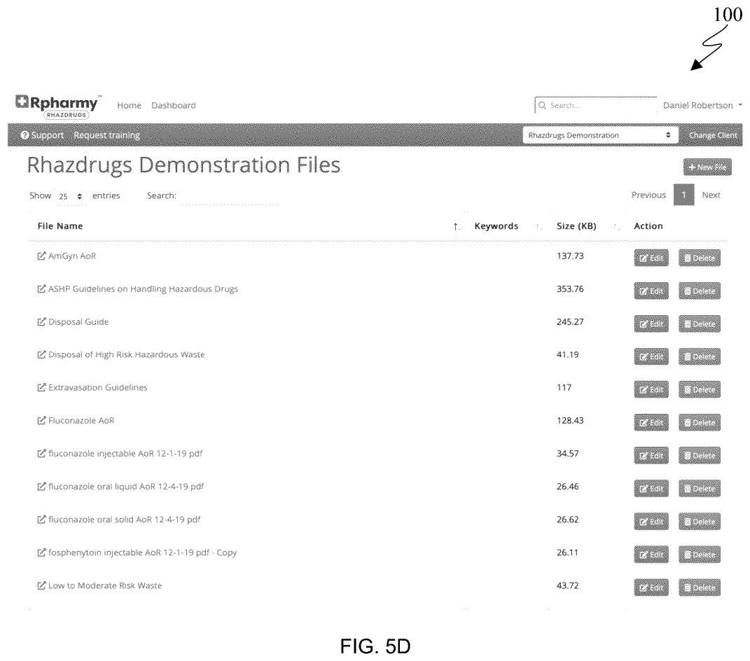

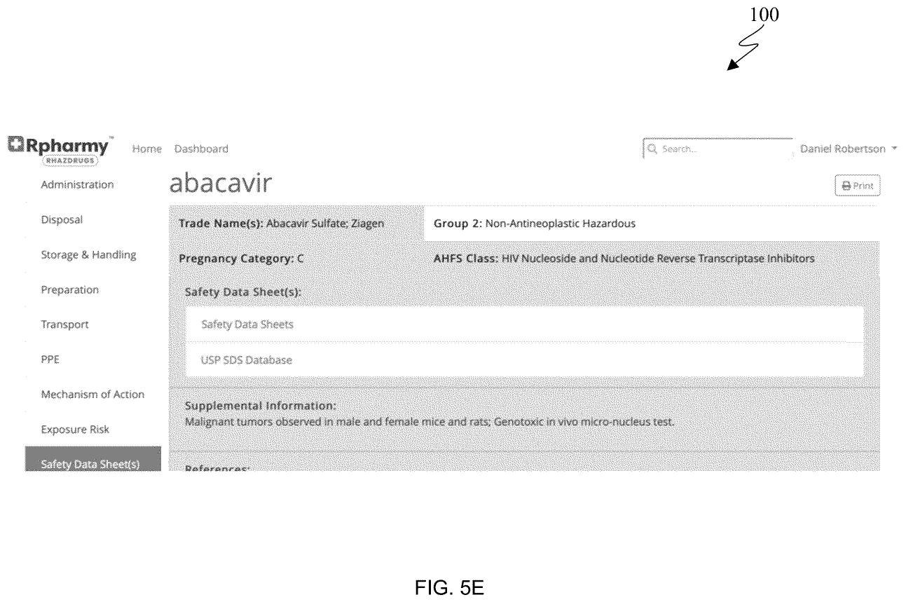

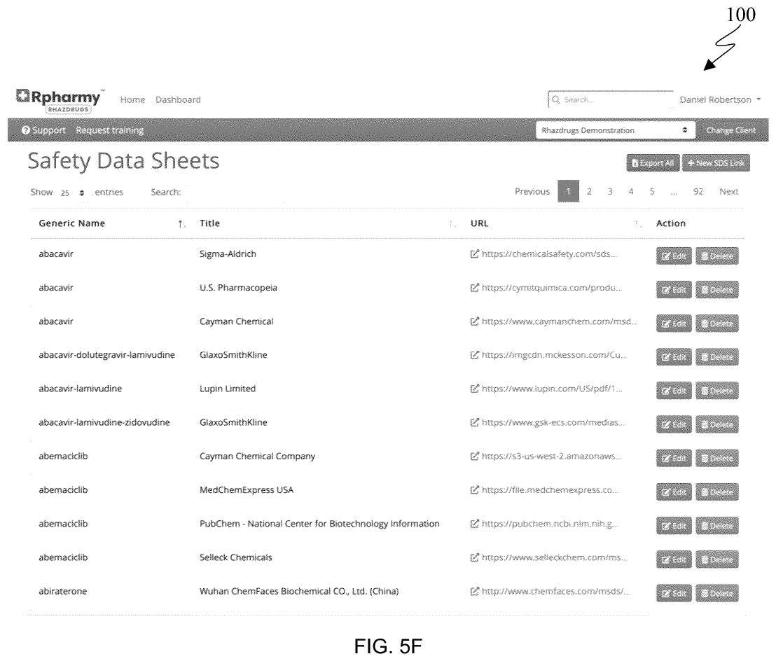

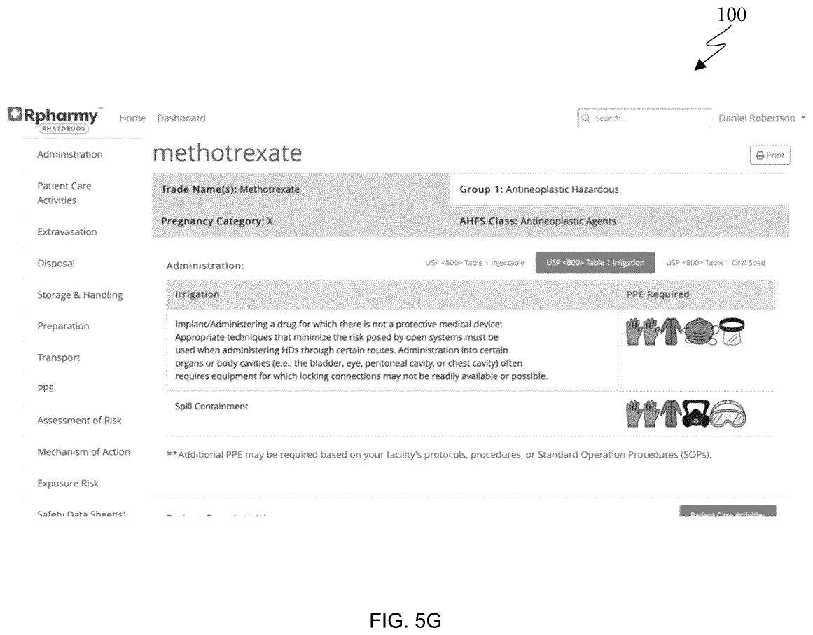

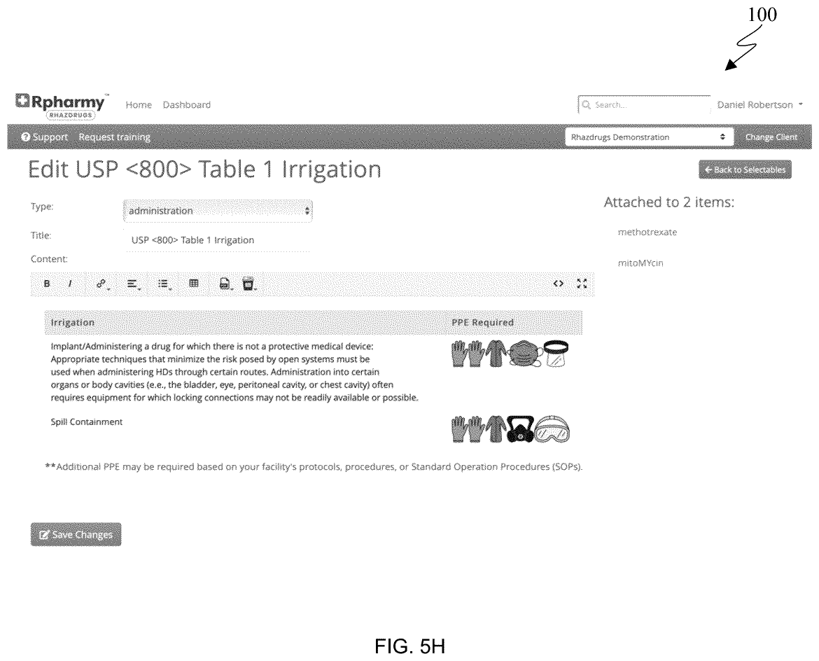

FIGS. 5A through 5H illustrate an example hazardous drug user interface in accordance with this disclosure. In particular, FIG. 5A illustrates an example approvals interface 500, FIG. 5B illustrates an example client drugs interface 502, FIG. 5C illustrates an example drugs interface 504, FIG. 5D illustrates an example files interface 506, FIG. 5E illustrates an example SDS tab 508 of the hazardous drug user interface, FIG. 5F illustrates an example SDS interface 510, FIG. 5G illustrates an example selectables interface 512, and FIG. 5H illustrates an example selectable editing interface 514. The embodiments of the approvals interface 500, client drugs interface 502, drugs interface 504, files interface 506, SDS tab 508, SDS interface 510, selectables interface 512, editing interface 514 illustrated in FIGS. 5A through 5H are for illustration only. FIGS. 5A through 5H do not limit the scope of this disclosure to any particular implementation of a hazardous drug user interface.

As shown in FIG. 5A, an example approvals interface 500 can indicate information stored in a custom hazardous formulary of a client has been reviewed by an approved user. In certain embodiments, the approval can be time-limited, such as a year. The administrative server can notify authorized users in advance when an approval approaches expiration. For example, the administrative server can email an authorized user a month in advance. The approvals can be stored in the approved_by field and the approved_date field of the client drugs data substructure 304 shown in FIG. 3C. The approvals interface 500 can also pull any other information from the client drugs data substructure 304 including a client drug ID field, a client drug client ID field, a client drug ID field, a client drug group field, a client drug AOR field, a client drug extravasation field, a client drug mechanism of action field, a client drug exposure risk field, a client drug SDS field, a client drug education field, a client drug training field, a client drug consent required field, a client drug approved by field, a client drug approved date field, a client drug references field, a client drug additional field, a client drug created at field, a client drug updated at field, etc.

As shown in FIG. 5B, an example client drugs interface 502 can indicate information about hazardous drugs added to a hospital hazardous drug data structure on a hospital hazardous drug database or on an administrative server. The client drugs interface 502 can also include custom information related to hazardous drugs altered by a client. Hazardous drugs can be linked to a client by a client ID field in the client drug data substructure. The client drug interface 502 can also pull or store information in the client drugs data substructure 304 including a client drug ID field, a client drug client ID field, a client drug ID field, a client drug group field, a client drug AOR field, a client drug extravasation field, a client drug mechanism of action field, a client drug exposure risk field, a client drug SDS field, a client drug education field, a client drug training field, a client drug consent required field, a client drug approved by field, a client drug approved date field, a client drug references field, a client drug additional field, a client drug created at field, a client drug updated at field, etc.

As shown in FIG. 5C, an example drugs interface 504 can display a master list of hazardous drugs. The drugs interface 504 can also include source information that a client can view. Any information updated in the drugs interface 504 can be automatically updated in a client drugs interface 502. The drugs interface 504 can pull or update information with the drugs data substructure 308 including a drugs ID field, a drugs multum ID field, a drugs generic name field, a drugs trade name field, a drugs group field, a drugs pregnancy category field, a drugs AHFS class field, a drugs extravasation field, a drugs patient care field, a drugs mechanism of action field, a drugs exposure risk field, a drugs SPS field, a drugs supplemental field, a drugs storage handling field, a drugs formulations field, a drugs BBX warning field, a drugs reference field, a drugs created at field, a drug updated at field, etc.

As shown in FIG. 5D, an example files interface 506 can allow files to be uploaded by a client to use in a client drug substructure or in a selectables substructure. Files can be linked to a client by a client ID field. Files in the files interface 506 can be associated with a files data substructure 314 that includes a files ID field, a files client ID field, a files name field, a files path field, a files keywords field, a files size field, a files created at field, a files updated at field, etc.

As shown in FIGS. 5E and 5F, an example SDS tab 508 and an example SDS interface 510 can be used to maintain a list of weblinks to publicly available safety data sheets for hazardous drugs in a master list. The SDS tab 508 and SDS interface 510 can also maintain weblinks to SDS for hazardous drugs stored in non-public databases, such as administrative database and hospital hazardous drug databases. The SDS tab 508 and SDS interface 510 can read or update information stored in the SDS data substructure including a SDS ID field, a SDS multum ID field, a SDS title field, a SDS URF field, a SDS created at field, a SDS updated at field, etc.

As shown in FIGS. 5G and 5H, an example selectables interface 512 and an example selectable editing interface 514 can display selectables from the selectables data substructure 318. Selectables are reusable blocks of information that can be assigned to certain sections in a client drug interface 502. The selectables can be grouped by type and can be selected for use in appropriate sections of the client drugs interface 502. Selectable can be linked to a client by a client ID and can be linked to many client drugs. The selectables interface 512 and the selectable editing interface 514 can read or update information from the selectables data substructure 318 including a selectables ID field, a selectables client ID field, a selectables type field, a selectables content field, a selectables created at field, a selectables updated at field, etc.

Although FIGS. 5A through 5H illustrate a hazardous drug user interface, various changes may be made to FIGS. 5A through 5H. For example, the number and placement of various components of the approvals interface 500, client drugs interface 502, drugs interface 504, files interface 506, SDS tab 508, SDS interface 510, selectables interface 512, editing interface 514 can vary as needed or desired. In addition, the approvals interface 500, client drugs interface 502, drugs interface 504, files interface 506, SDS tab 508, SDS interface 510, selectables interface 512, editing interface 514 may be used in any other suitable hazardous drug administrative process and is not limited to the specific processes described above.

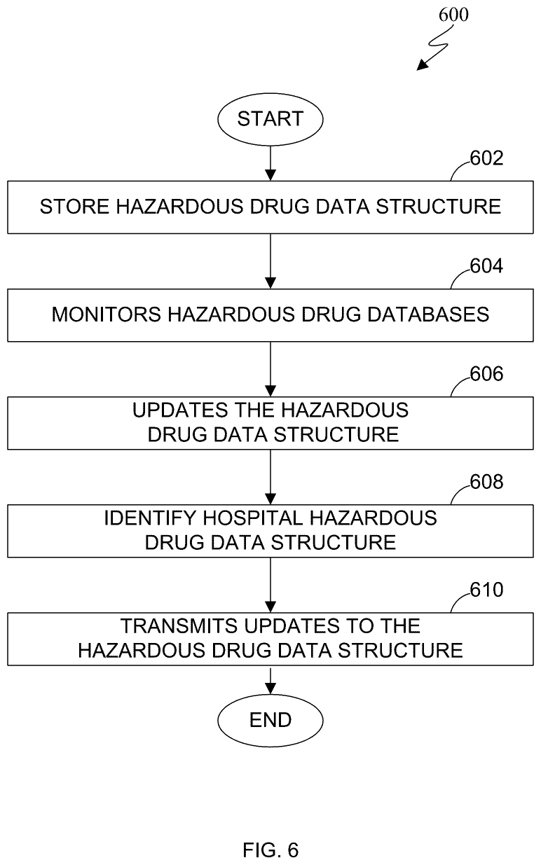

FIG. 6 illustrates an example method 600 for administering safety information for hazardous drugs according to this disclosure. For ease of explanation, the method 600 of FIG. 6 is described as being performed using the administrative server 101 of FIG. 1. However, the method 600 may be used with any other suitable system and any other suitable hazardous drug server or hazardous drug database.

As shown in FIG. 6, the administrative server 101 stores a hazardous drug data structure at step 602. The administrative server 101 can include a hardware processor 202 and a memory 204. The memory 202 of the administrative server 101 can store the hazardous drug data structure. An input device 208 coupled to the administrative server 101 can allow a user to enter information into the hazardous drug data structure 300. A display/audio output 212 of the administrative server 101 can output a display or audio related to the hazardous drug data structure 300. A communication interface 214 of the administrative server 101 can transmit or receive information related to the hazardous drug structure 300.

The hazardous drug data structure 300 can include a plurality of hazardous drug data substructures 302-322. The hazardous drug data structure 300 can include one or more of a clients data substructure 302, a client drug data substructure 304, a client drug selectable data substructure 306, a drugs data substructure 308, a users data substructure 310, a changelog data substructure 312, a files data substructure 314, an IP addresses data substructure 316, a selectables data substructure 318, an SDS data substructure 320, and a multum drugs data substructure 322.

Each of the plurality of hazardous drug data substructures includes different variable fields. The client data substructure 302 can store clients data including a client ID field, a client name field, a client slug field, a client active field, a client IP field, a client settings field, a client created at field, a client updated at field, etc. The client drug data substructure 304 can store client drug data including a client drug ID field, a client drug client ID field, a client drug ID field, a client drug group field, a client drug AOR field, a client drug extravasation field, a client drug mechanism of action field, a client drug exposure risk field, a client drug SDS field, a client drug education field, a client drug training field, a client drug consent required field, a client drug approved by field, a client drug approved date field, a client drug references field, a client drug additional field, a client drug created at field, a client drug updated at field, etc.

The client drug selectable data substructure 306 can store client drug selectable data including a client drug selectable ID field, a client drug selectable selectable ID field, a client drug selectable created at field, a client drug selectable updated at field, etc. The drugs data substructure 308 can store drug data including a drugs ID field, a drugs multum ID field, a drugs generic name field, a drugs trade name field, a drugs group field, a drugs pregnancy category field, a drugs AHFS class field, a drugs extravasation field, a drugs patient care field, a drugs mechanism of action field, a drugs exposure risk field, a drugs SPS field, a drugs supplemental field, a drugs storage handling field, a drugs formulations field, a drugs BBX warning field, a drugs reference field, a drugs created at field, a drug updated at field, etc.

The users data substructure 310 can store users data including a users ID field, a users name field, a users sub field, a users email field, a users remember token field, a users client ID field, a users created at field, a users updated at field, etc. The changelog data substructure 312 can store changelog data including a changelog ID field, a changelog user ID field, a changelog client ID field, a changelog model field, a changelog action field, a changelog message field, a changelog models field, a changelog created at field, a changelog updated at field, etc.

The files data substructure 314 can store files data including a files ID field, a files client ID field, a files name field, a files path field, a files keywords field, a files size field, a files created at field, a files updated at field, etc. The IP addresses data substructure 316 can store IP addresses data can include an IP addresses ID field, an IP addresses client ID field, an IP addresses IP field, an IP addresses created at field, an IP addresses updated at field, etc. The selectables data substructure 318 can store selectables data including a selectables ID field, a selectables client ID field, a selectables type field, a selectables content field, a selectables created at field, a selectables updated at field, etc.

The SDS data substructure 320 can store SDS data including a SDS ID field, a SDS multum ID field, a SDS title field, a SDS URF field, a SDS created at field, a SDS updated at field, etc. The multum drugs data substructure 322 can store multum drug data including a multum drug ID field, a multum drug generic name field, a multum drug trade name field, a multum drug pregnancy category field, a multum drug created at field, a multum drug updated at field, etc.

A first variable field of a first hazardous drug data substructure is linked to a first variable field of a second hazardous drug data substructure. Examples of linked variable fields can include links A-K shown in FIGS. 3A-3K.

An update to the first variable field of the first hazardous drug data substructure is also applied as an update to the first variable field of the second hazardous drug data substructure. For example, when a client ID field is updated in any of a clients data substructure 302, a client drugs data substructure 304, a changelog data substructure 312, a files data substructure 314, an IP addresses substructure 316, and a selectables data substructure 318, the other client ID fields can be automatically updated.

The administrative server 101 can store a local hospital hazardous drug structure for each of the hospital hazardous drug data structures located at a hospital hazardous drug database. Data structure 300 is an example of a data structure that can be duplicated for a master or administrative data structure and a hospital data structure. The hospital data structure can have a mirror data structure as a local hospital data structure located at the administrative server 101.

The administrative server 101 monitors hazardous drug databases for updates to the hazardous drug data structure at step 604. The administrative server 101 can monitor federal hazardous drug databases 102, state hazardous drug databases 104, local hazardous drug databases, other hazardous drug databases 108, and hazardous drug manufacturer servers/databases 120.

The administrative server 101 updates the hazardous drug data structure based on the updates from the monitored hazardous drug databases at step 606. When an update is detected on one or more of the federal hazardous drug databases 102, state hazardous drug databases 104, local hazardous drug databases, other hazardous drug databases 108, and hazardous drug manufacturer servers/databases 120 to specific hazardous drug info, the variable fields that correspond to the updated data in the hazardous drug data structure 300 are updated.

The administrative server 101 identifies hospital hazardous drug data structures that are linked to the hazardous drug data structure at step 608. Hospital hazardous drug data structures can be traversed or a local hospitalized data structure corresponding to the hospital hazardous drug data structure can be traversed. Identifying the hospital hazardous drug data structures that are linked to the hazardous drug data structure can include determining a hazardous drug data substructure that is updated based on the monitored hazardous drug databases and identifying a hospital hazardous drug data structure that includes the updated hazardous drug data substructure.

The administrative server 101 transmits the updates to the hazardous drug data structure that correspond to the identified hospital hazardous drug data structures at step 610. This ensures that the users and clients have the most updated information regrading administration and handling of hazardous drugs.

Although FIG. 6 illustrates one example of a method 600 for administering safety information for hazardous drugs, various changes may be made to FIG. 6. For example, while shown as a series of steps, various steps in FIG. 6 may overlap, occur in parallel, or occur any number of times.

In some embodiments, various functions described in this patent document are implemented or supported by a computer program that is formed from computer readable program code and that is embodied in a computer readable medium. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

It may be advantageous to set forth definitions of certain words and phrases used throughout this patent document. The terms “include” and “comprise,” as well as derivatives thereof, mean inclusion without limitation. The term “or” is inclusive, meaning and/or. The phrase “associated with,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

The description in the present application should not be read as implying that any particular element, step, or function is an essential or critical element that must be included in the claim scope. The scope of patented subject matter is defined only by the allowed claims. Moreover, none of the claims invokes 35 U.S.C. § 112(f) with respect to any of the appended claims or claim elements unless the exact words “means for” or “step for” are explicitly used in the particular claim, followed by a participle phrase identifying a function.

Although the present disclosure has been described with exemplary embodiments, various changes and modifications may be suggested to one skilled in the art. It is intended that the present disclosure encompass such changes and modifications as fall within the scope of the appended claims. None of the description in this application should be read as implying that any particular element, step, or function is an essential element that must be included in the claims scope. The scope of patented subject matter is defined by the claims.

What is claimed is:

1. A hazardous drug administrative server comprising:
a memory configured to store a hazardous drug data structure; and
a processor operably coupled to the memory, the processor configured to:
monitor hazardous drug databases for updates to the hazardous drug data structure;
update the hazardous drug data structure based on the updates from the monitored hazardous drug databases;
identify hospital hazardous drug data structures that are linked to the hazardous drug data structure; and
transmit the updates to the hazardous drug data structure that correspond to the identified hospital hazardous drug data structures to a hospital server.

2. The hazardous drug administrative server of claim 1, wherein the memory is further configured to store a local hospital hazardous drug structure for each of the hospital hazardous drug data structures located at a hospital hazardous drug database.

3. The hazardous drug administrative server of claim 2, wherein, to identify the hospital hazardous drug data structures that are linked to the hazardous drug data structure, the processor is configured to:
determine a hazardous drug data substructure that is updated based on the monitored hazardous drug databases, and
identify a hospital hazardous drug data structure that includes the updated hazardous drug data substructure.

4. The hazardous drug administrative server of claim 1, wherein the hazardous drug data structure includes a plurality of hazardous drug data substructures.

5. The hazardous drug administrative server of claim 4, wherein each of the plurality of hazardous drug data substructures includes different variable fields.

6. The hazardous drug administrative server of claim 4, wherein a first variable field of a first hazardous drug data substructure is linked to a first variable field of a second hazardous drug data substructure.

7. The hazardous drug administrative server of claim 6, wherein an update to the first variable field of the first hazardous drug data substructure is also applied as an update to the first variable field of the second hazardous drug data substructure.

8. A method of administering hazardous drug data, the method comprises:
storing a hazardous drug data structure;
monitoring hazardous drug databases for updates to the hazardous drug data structure;
updating the hazardous drug data structure based on the updates from the monitored hazardous drug databases;
identifying hospital hazardous drug data structures that are linked to the hazardous drug data structure; and
transmitting the updates to the hazardous drug data structure that correspond to the identified hospital hazardous drug data structures to a hospital server.

9. The method of claim 8, further comprising:
storing a local hospital hazardous drug structure for each of the hospital hazardous drug data structures located at a hospital hazardous drug database.

10. The method of claim 9, wherein, identifying the hospital hazardous drug data structures that are linked to the hazardous drug data structure, the method comprises:
determining a hazardous drug data substructure that is updated based on the monitored hazardous drug databases, and
identifying a hospital hazardous drug data structure that includes the updated hazardous drug data substructure.

11. The method of claim 8, wherein the hazardous drug data structure includes a plurality of hazardous drug data substructures.

12. The method of claim 11, wherein each of the plurality of hazardous drug data sub structures includes different variable fields.

13. The method of claim 11, wherein a first variable field of a first hazardous drug data substructure is linked to a first variable field of a second hazardous drug data substructure.

14. The method of claim 13, wherein an update to the first variable field of the first hazardous drug data substructure is also applied as an update to the first variable field of the second hazardous drug data substructure.

15. A hazardous drug system comprising:
hazardous drug databases configured to store hazardous drug information; and
a hazardous drug administrative server communicatively coupled to the hazardous drug databases, the hazardous drug administrative server is configured to:
store a hazardous drug data structure,
monitor hazardous drug databases for updates to the hazardous drug data structure,
update the hazardous drug data structure based on the updates from the monitored hazardous drug databases,
identify hospital hazardous drug data structures that are linked to the hazardous drug data structure, and
transmit the updates to the hazardous drug data structure that correspond to the identified hospital hazardous drug data structures to a hospital server.

16. The hazardous drug system of claim 15, wherein the hazardous drug administrative server is further configured to store a local hospital hazardous drug structure for each of the hospital hazardous drug data structures located at a hospital hazardous drug database.

17. The hazardous drug system of claim 16, wherein, to identify the hospital hazardous drug data structures that are linked to the hazardous drug data structure, the hazardous drug administrative server is configured to:
determine a hazardous drug data substructure that is updated based on the monitored hazardous drug databases, and
identify a hospital hazardous drug data structure that includes the updated hazardous drug data substructure.

18. The hazardous drug system of claim 15, wherein the hazardous drug data structure includes a plurality of hazardous drug data substructures.

19. The hazardous drug system of claim 18, wherein each of the plurality of hazardous drug data substructures includes different variable fields.

20. The hazardous drug system of claim 18, wherein:
a first variable field of a first hazardous drug data substructure is linked to a first variable field of a second hazardous drug data substructure, and
an update to the first variable field of the first hazardous drug data substructure is also applied as an update to the first variable field of the second hazardous drug data substructure.
